(19) United States
(12) Patent Application Publication
(10) Pub. No.: US 2010/0015050 A1
(43) Pub. Date: Jan. 21, 2010

(54) PEG AND TARGETING LIGANDS ON NANOPARTICLE SURFACE

(75) Inventors: Jayanth Panyam, Novi, MI (US); Yogesh Patil, Troy, MI (US); Ayman Khdair, Dearborn, MI (US)

Correspondence Address: C. Rachal Winger;c/o OCIP Group, 1900 Main Street, Suite 600, Irvine, CA, 92614-7319, US

(73) Assignee: WAYNE STATE UNIVERSITY, Detroit, MI (US)

(21) Appl. No.: 12/519,590

(22) PCT Filed: Dec. 20, 2007
(86) PCT No.: PCT/US07/88454

§ 371 Date: Jun. 17, 2009

Related U.S. Application Data
(60) Provisional application No. 60/871,404, filed on Dec. 21, 2006.

(51) Int. Cl. A61K 31/337; A61K 47/34; A61K 47/48; A61P 35/00; A61K 51/04; A61K 31/567; A61K 38/02; A61K 31/4985; A61K 38/54; A61K 49/10

(52) U.S. Cl. 424/1.65; 514/772.3; 514/772.7; 514/449; 514/178; 514/2; 514/249; 424/94.3; 424/9.3

(57) ABSTRACT

Provided are compositions of nanoparticles, PEG and targeting moieties. The compositions are useful in treating tumors, imaging the particles in tissues, and in targeting therapeutic agents to specific tissues and locations in a patient. Also provided are methods of preparing and methods of using the compositions.

### STATEMENT OF GOVERNMENT INTEREST

This work was supported in part by grant number 7R21 CA116641-02 from the National Institutes of Health. The government may have certain rights in this invention.

### TECHNICAL FIELD

The present invention is directed to compositions of nanoparticles, PEG and targeting moieties.

### BACKGROUND OF THE INVENTION

The term “nanoparticle” has been used to refer to nanometer-size devices consisting of a matrix of dense polymeric network (also known as nanospheres) and those formed by a thin polymeric envelope surrounding a drug-filled cavity (nanocapsules) (Garcia-Garcia et al., Int. J. Pharm., 298:274-92, 2005). Nanoparticles can penetrate into small capillaries, allowing enhanced accumulation of the encapsulated drug at target sites (Calvo et al., Pharm. Res. 18:1157-66; 2001). Nanoparticles can passively target tumor tissue through enhanced permeation and retention effect (Monsky et al., Cancer Res. 59:4129-35, 1999; Stroh et al., Nat. Med. 11:678-82, 2005). Nanoparticles can be delivered to distant target sites either by localized catheter-based infusion (Panyam et al., J. Drug Target. 10:515-523, 2002) or by attaching a ligand to nanoparticle surface that has affinity for a specific tissue (Shenoy et al., Pharm. Res. 22:2107-14, 2005). Because of sustained release properties, nanoparticles can prolong the availability of the encapsulated drug at the target site, resulting in greater and sustained therapeutic effect (Panyam and Labhasetwar, Adv. Drug Deliv. Rev. 55:329-47, 2003).

Chemotherapy resistance is a frequent phenomenon in cancer cells (Stein et al., Curr. Drug Targets 5:333-46, 2004). The significance of this problem is highlighted by the estimations that up to 500,000 new cases of cancer each year will eventually exhibit drug-resistant phenotype (Shabbits et al., Expert Rev. Anticancer Ther. 1:585-94, 2001). There is a need in the art for improved delivery of cancer therapeutics.

A limitation for any nanoparticulate system used in systemic drug delivery is their rapid clearance from the circulation by the reticuloendothelial system (RES) (Owens and Peppas, Int. J. Pharm. 307:93-102, 2006). The RES comprises of a group of cells having the ability to take up and sequester particles, including macrophages or macrophage precursors, specialized endothelial cells lining the sinusoids of the liver, spleen, and bone marrow, and reticular cells of lymphatic tissue (macrophages) and of bone marrow (fibroblasts) (Frank and Fries, Immunol. Today 12:322-6, 1991). Rapid uptake of the drug carrier by RES reduces drug's availability at the target site. RES clearance can be reduced by coating nanoparticles with hydrophilic polymers such as poly(ethylene glycol) (PEG) (Owens and Peppas, Int. J. Pharm. 307:93-102, 2006).

“PEGylation” refers to the decoration of particle surface by covalently grafting or adsorbing of PEG chains. The purpose of PEG chains is to create a barrier to the adhesion of opsonins present in the blood, so that delivery systems can remain longer in circulation, invisible to phagocytic cells (Kommareddy et al., Technol. Cancer Res. Treat. 4:615-26, 2005). While several theories have been proposed to explain the mechanism of PEGylation (Moghimi and Szebeni, Prog. Lipid Res. 42:463-78, 2003), the most widely accepted theory is based on the hypothesis that PEGylation adds protein resistant properties to materials (Jeon et al., J. Coll. Interface Sci. 142:149-158, 1991). This theory suggests that the hydrophilic and flexible nature of PEG chains allows them to take on an extended conformation when free in solution.

When opsonins are attracted to the surface of the particle by van der Waals and other forces, they encounter the extended PEG chains and begin to compress them. This compression then forces the PEG chains into a more condensed and higher energy conformation. This change in conformation creates an opposing repulsive force that, when great enough, can completely balance and/or overpower the attractive force between the opsonin and the particle surface. For effective blocking of opsonins to occur, the surface coating layer needs to exceed a minimum layer thickness. The layer thickness is governed by factors such as PEG molecular weight, surface chain density, and conformation. Most studies indicate that a PEG molecular weight of 2000 Da or greater is required to achieve stealth properties (Storm et al., Adv. Drug Del. Rev. 17:31-48, 1995). This may be due in part to the increased chain flexibility of higher molecular weight PEG polymers (Gref et al., Adv. Drug Del. Rev. 16:215-233, 1995; Leroux et al., Life Sci. 57:695-703, 1995; Peracchia et al., Life Sci. 61:749-61, 1997).

Previous attempts to introduce PEG and targeting ligands nanoparticles have utilized either surface adsorption of PEG-containing block copolymers/ligands (Cho et al., Macromol. Biosci. 5:512-519, 2005) or chemical coupling of PEG/ligands to the surface of nanoparticles (Sahoo and Labhasetwar, Mol. Pharm. 2:373-83, 2005). Surface adsorption is a simple way of modifying nanoparticle surface and is independent of nanoparticle composition. However, surface adsorption relies on weak physical forces between nanoparticle surface and the surface-modifying agent. This contributes to easy desorption of both PEG and targeting ligand from nanoparticle surface in a biological environment. Covalent coupling of PEG/ligand to nanoparticle surface ensures that PEG and ligand are firmly attached to nanoparticle surface. However, chemical conjugation has a number of disadvantages: (1) functional groups are not always available on nanoparticle surface for attaching PEG/ligands, (2) material used in nanoparticle formulation (polymer, therapeutic agent) may not be compatible with solvents used in chemical conjugation, (3) there is a possibility of leaching of the nanoparticle payload during the synthesis step, and (4) new synthetic procedures may have to be developed for each new nanoparticle-ligand combination.

An alternative approach that has been investigated is the use of PEGylated polymers in nanoparticle formulation. For example, instead of chemically attaching PEG chains to nanoparticles prepared from polylactide (PLA) polymer, nanoparticles have been prepared using PLA-PEG polymer (Avgoustakis, Curr. Drug Deliv. 1:321-33, 2004). While this results in PLA nanoparticle with some PEG on the surface, the physico-chemical properties (drug encapsulation, release, biological half-life) of these nanoparticles are markedly different from PLA nanoparticles. For example, PLA nanoparticles, in general, show significantly more sustained release of the encapsulated therapeutic agent than PLA-PEG nanoparticles (Dong and Feng, J. Biomed. Mater Res. A 78:12-9, 2006).

### SUMMARY OF THE INVENTION

Provided is a method of treating a tumor in a subject, the method comprising contacting a subject in need thereof with a nanoparticle comprising at least one polymer and at least one therapeutic agent joined thereto, under suitable conditions such that at least one tumor-related effect occurs.

The tumor-related effect may be selected from the group consisting of: decrease in tumor size, decrease in tumor cell proliferation, decrease in tumor cell metastasis, decrease in tumor vasculature, decrease in tumor angiogenesis, decrease in tumor blood flow, increase in cell differentiation, increase in tumor cell apoptosis, and increase in tumor cell necrosis.

The suitable conditions comprise a sustained time period of at least 1 day, at least 2 days, at least 5 days, at least 10 days, at least 20 days, at least 30 days, at least 45 days, and at least 60 days.

The polymer may be selected from the group consisting of: aliphatic polyesters; poly(glycolic acid); poly(lactic-co-glycolic acid); poly(caprolactone glycolide); poly(lactic acid); polylactide (PLA); poly-L(lactic acid); poly-D(lactic acid); poly(caprolactone lactide); poly(lactide glycolide), poly(lactic acid ethylene glycol)); poly(ethylene glycol); poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides; poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; and biodegradable polyesters.

The therapeutic agent is selected from the group consisting of: a polysaccharide, a peptide, a polypeptide, a nucleic acid, a vitamin, a mineral, a vaccine, a cytokine, an apoptotic agent, a cytotoxic agent, photosensitizer, and a pharmaceutical drug. The therapeutic agent can comprise paclitaxel, dexamethasone, heat-shock protein 70, Bcl-2, Bcl-xl, or folic acid.

The nanoparticle may further comprise a detection agent joined thereto, wherein the detection agent is selected from the group consisting of: a magnetic compound, a paramagnetic compound, a fluorophore, a radio-isotope, and an enzyme. The nanoparticle may further comprise a functional group joined thereto, wherein the functional group is selected from the group consisting of: alkane, alkene, alkyne, amide, amine, imide, phosphine, maleimide, phosphodiester, phosphonic acid, phosphate, sulfide, imidazole and oxazole.

Also provided is a therapeutic composition comprising a nanoparticle, and at least one therapeutic agent joined thereto wherein the therapeutic agent confers a sustained biological or chemical effect over a time period. The time period may be selected from the group consisting of: at least 1 day, at least 2 days, at least 5 days, at least 10 days, at least 20 days, at least 30 days, at least 40 days, and at least 60 days.

A process of making a nanoparticle composition comprising a first step of emulsifying at least one first agent in the presence of at least one first polymer and at least one first solvent, thereby forming a water-in-oil emulsion; and a second step of emulsifying the water-in-oil emulsion with at least one second polymer, at least one second solvent, and at least one second agent wherein the first and second agents are the same or different and are selected from the group consisting of a therapeutic agent, a diagnostic agent, and a detection agent; thereby making a nanoparticle composition. In preferred embodiments, the process results in the agent(s) joined or conjugated to the polymer-based nanoparticles.

In the process for making the nanoparticles, the first polymer may comprises poly(lactic co-glycolic acid) (PLGA), the first solvent may comprise polyvinyl alcohol, the first agent may comprise paclitaxel, dexamethasone, a heat-shock protein, Bcl-2, Bcl-xl, or folic acid, the second polymer may comprise polylactide (PLA) or polyethylene glycol (PEG), the second solvent may comprise methanol, and the second agent may comprise folic acid.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a proposed mechanism of efficacy with dual-agent nanoparticles, in accordance with an embodiment of the invention. Inhibition of P-gp expression is shown as an example.

FIG. 2 depicts that nanoparticle encapsulated paclitaxel is effective in drug-sensitive (A) but not drug-resistant cells (B), in accordance with an embodiment of the invention. Drug sensitive (MCF-7) and drug-resistant (NCl/ADR-RES) cells were plated in 96-well plates at a density of 5000 cells/well/0.1 mL. Cells were then treated with paclitaxel (Pac) in solution (100 nM) or equivalent in nanoparticles (NP). Some resistant cells were treated with paclitaxel solution in the presence of verapamil (100 μM). Untreated cells and cells treated with empty nanoparticles were used as controls. Cytotoxicity was followed using a standard MTS assay (see Methods). The medium was changed on day 2 and every other day thereafter, and no further dose of paclitaxel or verapamil was added. Data as mean±SD, n=6 wells. * P<0.05.

FIG. 3 depicts that inhibition of P-gp overcomes resistance to nanoparticle-encapsulated paclitaxel, in accordance with an embodiment of the invention. NCl/ADR-RES cells were plated in 96-well plates at a density of 5000 cells/well/0.1 mL. Cells were then treated with paclitaxel (Pac, 100 nM) in nanoparticles (NP). Some cells were treated with paclitaxel nanoparticles in the presence of single or multiple dose verapamil (100 μM). Untreated cells were used as controls. Cytotoxicity was followed as a function of time using a standard MTS assay. The medium was changed on day 2 and every other day thereafter, and no further dose of drug was added. In some cells, verapamil was added every time the media was changed (multiple dosing). Data as mean±SD, n=6 wells. * P<0.05 compared to controls. # P<0.05 compared to single dose verapamil.

FIG. 4 depicts that P-gp does not affect nanoparticle uptake and retention (A) but reduces paclitaxel accumulation (B), in accordance with an embodiment of the present invention. NCl/ADR-RES cells were plated in 24-well plates at a density of 50,000 cells/well/1 mL. A suspension of nanoparticles (NP) loaded with paclitaxel (Pac) and 6-coumarin was prepared in regular serum-containing growth medium (100 μg/mL, 1 mL), and was added to each well in the presence or absence of verapamil (100 nM). The medium was changed on day 2 and every other day thereafter, and no further dose of drug was added. Cells were harvested at different time intervals, and lysed using cell culture lysis reagent (Promega). Nanoparticle uptake was quantified by measuring 6-coumarin concentration in the cell lysates by HPLC, as described previously (J. Panyam, et al. Fluorescence and electron microscopy probes for cellular and tissue uptake of poly(D,L-lactide-co-glycolide) nanoparticles. Int J Pharm 262: 1-11 (2003). Paclitaxel concentration was determined by HPLC, as described in the Examples. Paclitaxel concentration in cells treated with paclitaxel nanoparticles alone was below the limit of detection. Nanoparticle and paclitaxel concentrations were normalized to total cell protein. Data as mean±SD.

FIG. 5 depicts in vitro release of paclitaxel (A) and siRNA (B) from nanoparticles, in accordance with an embodiment of the invention. (A) About 0.5 ml of nanoparticle suspension in PBS (2 mg/ml) containing 0.1% w/v Tween 80 in dialysis tube (Pierce; 2000 Da MWCO) was incubated with 10.5 ml of PBS containing 0.1% w/v Tween 80 in a 15-ml Eppendorf tube at 37° C., and shaken at 100 rpm. Samples of dialysate were taken at different time intervals, and paclitaxel concentration was determined by HPLC. (B) About 160 μg of nanoparticles was incubated with 0.2 ml of nuclease-free PBS in a 2-ml Eppendorf tube at 37° C., and shaken at 100 rpm. At different time points, nanoparticle suspension was centrifuged, and siRNA released in the supernatant was determined by Picogreen assay. Data represented as mean±SD, n=3.

FIG. 6 depicts that dual-agent nanoparticles overcome resistance to paclitaxel, in accordance with an embodiment of the invention. Drug-resistant (NCl/ADR-RES) cells were plated in 96-well plates at a density of 5000 cells/well/0.1 mL. Cells were then treated with dual-agent nanoparticles releasing 0.3 ng/day/8 μg siRNA and 7 ng/day/8 μg paclitaxel. Cells treated with non-targeted (scrambled) siRNA and untreated cells were used as controls. Cytotoxicity was followed using a standard MTS assay. The medium was changed on day 2 and every other day thereafter, and no further dose of drug was added. Data as mean±SD, n=6 wells. * P<0.05 compared to controls.

FIG. 7 depicts nanoparticle formulations with different drug release rates, in accordance with an embodiment of the invention. Nanoparticles were formulated with polymers that differed in (A) lactide-to-glycolide ratio, (B) molecular weight, or (C) end-group chemistry, and the in vitro release of encapsulated dexamethasone was studied in a side-by-side diffusion chamber. Nanoparticle dispersion in PBS was added to the donor chamber, while buffer was added to the receiver chamber. Drug released into the receiver buffer was analyzed by measuring the radioactivity of tritiated drug. Data as mean±SD, n=3.

FIG. 8 depicts a correlation between dose of drug released and therapeutic efficacy, in accordance with an embodiment of the invention. Nanoparticle formulations with different drug release rates were formulated by emulsion solvent evaporation technique. In vitro drug release from the formulations was studied as described for FIG. 7. Data represented as mean±SD, n=3. The two formulations were investigated for cytotoxicity in vascular smooth muscle cells. Cells were plated in 96-well plates at a density of 5000 cells/well/0.1 mL. A nanoparticle suspension prepared in regular serum-containing growth medium (600 μg/mL, 0.1 mL) was added to each well. Drug solution in growth medium (25 μg/mL) was also added to some of the wells. Untreated cells were used as controls. Cytotoxicity was determined using a standard MTS assay. The medium was changed on day 2 and every other day thereafter, and no further dose of drug was added. Data as mean±SD, n=6. To confirm that differences in cytotoxicity with two formulations were due to the differences in the dose of the drug released intracellularly, the intracellular drug accumulation following treatment was followed with ³H-dexamethasone in solution or in nanoparticles. Data represented as mean±SEM, n=3. Cells were plated at 100,000 cells/well/2 ml in 6-well plates. Drug encapsulated in nanoparticles (600 μg of nanoparticles/ml, 2 ml) or in solution (25 μg/ml, 2 ml) was added to each well. Medium was changed on day 2 and every other day thereafter, and no further dose of the drug was added. Radioactivity in the extracts was measured using a scintillation counter.

FIG. 9 depicts the effect of folic acid and PEG incorporation on tumor-targeting of nanoparticles, in accordance with an embodiment of the invention. Tumors were initiated in female Balb/c mice by subcutaneous injection of JC cell suspension (106 cells) in the right hind quarter. Mice that developed tumors of at least 100 mm³ volume were injected intravenously with treatments equivalent to 2 mg/kg dose of nanoparticles. Mice were euthanized at the end of 6 hrs and tumors were collected. Nanoparticle (NP) concentration in tumors was analyzed by HPLC and was normalized to the weight of the organ. 0/100—no folate and only PEG; 50/50—folate-PEG and PEG in 50:50 ratio; 100/0—only PEG-folate (n=5).

FIG. 10 describes the effect of folic acid or biotin conjugation on nanoparticle uptake in the four different cancer cell lines. Folic acid or biotin conjugation increases nanoparticle uptake in these cells. When excess free folic acid or biotin was added, this enhancement was diminished because of competition between free folic acid and folic acid-conjugated nanoparticles for folic acid receptors.

FIG. 11 shows the effect of folic acid conjugation on nanoparticle retention in NCl/ADR cancer cell line. As the graph indicates, folic acid conjugation not only increased the amount of nanoparticles taken up by cells (0 hrs) but also the amount that is retained in the cells over a course of 120 minutes.

FIG. 12A illustrates the effect of folic acid and biotin conjugation on in vitro cytotoxicity of paclitaxel in breast cancer cell line MCF-7. Conjugation of biotin and paclitaxel on nanoparticles increased the cytotoxicity (decreased % viability) of nanoparticle encapsulated paclitaxel. This effect was sustained over three days of the study (FIG. 12B).

FIG. 13A shows the behavior of amphiphilic diblock copolymer in an oil/water biphasic system. FIG. 13B shows the introduction of PLA-PEG and PLA-PEG-ligand conjugate during the emulsification step results in nanoparticles with PEG and PEG-ligand on nanoparticle surface.

FIGS. 14A and B shows surface plasmon resonance analysis of functionalized nanoparticles. FIG. 14A shows biotin conjugated nanoparticles on streptavidin surface. FIG. 14B shows folic acid conjugated nanoparticles on anti-folic acid monoclonal antibody coated surface.

FIG. 15A shows that incorporation of PEG on nanoparticle surface increases plasma half-life. FIG. 15B shows that incorporation of folic acid enhances tumor accumulation of PLGA nanoparticles.

FIG. 16A shows that incorporation of PEG-folic acid and/or PEG-biotin on nanoparticle surface results in enhanced tumor growth inhibition. FIG. 16B shows animal survival following treatment with nanoparticle-encapsulated paclitaxel.

FIG. 17 shows ¹H NMR spectrum of PLA-PEG conjugated PLGA NP (PEG:CH₂ at 3.6 ppm; PLA:CH at 1.62 ppm and CH₃ at 5.22 ppm).

### DESCRIPTION OF THE INVENTION

One skilled in the art will recognize many methods and materials similar or equivalent to those described herein, which could be used in the practice of the present invention. Indeed, the present invention is in no way limited to the methods and materials described.

In order to overcome the disadvantages of existing methods to introduce PEG and ligands on nanoparticle surface, the present disclosure provides a novel technique to anchor PEG and PEG-folate conjugate on the surface of nanoparticles. This technique relies on the interfacial activity of PEG-X block copolymer conjugate, where X is any hydrophobic polymer (example, polylactide, polypropylene oxide, etc). Most nanoparticle formulations involve an emulsion step in the preparation. Following the formation of the emulsion, a methanol solution of PEG-containing block copolymer (for example PLA-PEG (1000/5000 Da) block copolymer, with or without conjugated ligand (folic acid, for example), is added to the emulsion. PLA-PEG is a surface active block copolymer, composed of hydrophobic PLA chains and hydrophilic PEG chains. Addition of the block copolymer to the emulsion results in the hydrophobic polylactide chain inserting itself into the oil phase and the hydrophilic PEG (or PEG-folate) chain remaining in the outer most aqueous phase. This results in nanoparticles that contain PEG (or folate-PEG) chains on the surface. Because this method relies only on the interfacial activity of the copolymer, the technique is independent of the polymer used for nanoparticle formulation or the targeting ligand that is being investigated.

Folic acid is an appealing ligand for targeted cellular drug delivery. Folate receptor is overexpressed on many human cancer cell surfaces (Turk et al., Arthritis Rheum. 46:1947-55, 2002). Thus, folic acid conjugates can be used to specifically target cancer cells. Although the reduced folate carrier is present in virtually all cells, folate-conjugates are not substrates and are taken up only by cells expressing functional folate receptors (Hilgenbrink and Low, J. Pharm. Sci. 94:2135-46, 2005). Folic acid conjugation allows endocytic uptake of the conjugated carrier via the folate receptor, resulting in higher cellular uptake of the encapsulated drug (Mansouri et al., Biomaterials, 2005). The high affinity of folic acid to its receptor (binding constant ˜1 nm) and folate's small size allow its use for specific cell targeting (Lee and Low, J. Biol. Chem. 269:3198, 1994). The ability of folic acid to bind its receptor is not altered by covalent conjugation to delivery systems (Lee and Low, J. Biol. Chem. 269:3198, 1994). Previous studies have shown selective delivery of drugs using folate-linked delivery systems to cancer cells overexpressing folate receptors. (Gabizon et al. Adv. Drug Del. Rev. 56:1177, 2004; Hilgenbrink and Low, J. Pharm. Sci. 94:2135-46, 2005; Kukowska-Latallo et al., Cancer Res. 65:5317-24, 2005; Paranjpe et al., J. Control Release 100:275, 2004; Rossin et al., J. Nucl. Med. 46:1210-8, 2005; Santra et al., J. Nanosci. Nanotechnol. 5:899-904, 2005; Wang and Hsiue, Bioconjug. Chem. 16:391-6, 2005).

Kartner and coworkers first demonstrated correlation between increased expression of P-gp in tumor cells with the development of multidrug resistance (MDR) (Kartner et al., Science 221:1285-8, 1983). This was followed by Chen et al., who described the sequence of the MDR1 cDNA and its homology to two bacterial transporters, thereby defining the first member of the ATP-binding Cassette (ABC) transporter family (Chen et al., Cell 47:381-9, 1986). It was later shown that expression of a full length cDNA for the human MDR1 gene confers drug resistance in tumor cells, confirming the role of MDR1 gene in drug resistance (Ueda et al., P.N.A.S. USA 84:3004-8, 1987). Since then, 48 human ABC genes have been identified and their roles in drug transport investigated (Dean et al., Genome Res. 11:1156-66, 2001). Of these, P-gp is one of the most consistently overexpressed transporters in drug resistant tumors (Gottesman, Annu. Rev. Med. 53:615-27, 2002). Evidence for the role of P-gp in clinical tumor resistance was provided by studies that demonstrated P-gp expression in about 40% of breast cancer samples and its correlation with decreased treatment response (Trock et al., J. Natl Cancer Inst. 89:917-31, 1997). Recent evidence further confirms this observation, and suggests that pretreatment P-gp expression is a strong predictor for clinical response to drug therapy (Chintamani et al., World J. Surg. Oncol. 3:61, 2005; Clarke et al., Semin. Oncol. 32:S9-15, 2005; Raspollini et al., Int. J. Gynecol. Cancer 15:255-60, 2005; Robey et al., Clin. Cancer Res. 12:1547-55, 2006).

Expression of P-gp leads to energy-dependent drug efflux and reduction in intracellular drug concentration. While the exact mechanism by which P-gp interacts with its substrate is not fully understood, it is thought that binding of a substrate to the high-affinity binding site results in ATP hydrolysis, causing a conformational change that shifts the substrate to a lower affinity binding site and then releases it into the extracellular space or outer leaflet of the membrane (Sauna et al., J. Bioenerg. Biomembr. 33:481-91, 2001). Whether P-gp extracts its substrate from the cytoplasm (Altenberg et al., P.N.A.S. USA 91:4654-4657, 1994) or from within the membrane (‘vacuum cleaner’ hypothesis) is not clear, but recent evidence suggests that substrates diffuse from the lipid bilayer into the drug-binding pocket located in a hydrophobic environment (Loo and Clarke, Biochem. Biophys. Res. Commun. 329:419-422, 2005; Lugo and Sharom, Biochem. 44:643-655, 2005). P-gp overexpression confers resistance to drugs through mechanisms not directly related to transport. For example, overexpression of P-gp confers resistance to complement-mediated cytotoxicity due to delayed deposition of complement on the plasma membrane (Weisburg et al., J. Exp. Med. 183:2699-704, 1996; Weisburg et al., J. Biol. Chem. 274:10877-88, 1999). Also, P-gp over-expressing cells are less sensitive to multiple forms of caspase-dependent cell death, including those mediated by Fas ligand (Ruefli et al., Cell Death Differ. 9:1266-72, 2002) and serum withdrawal (Robinson et al., Biochem. 36:11169-78, 1997). Levchenko and coworkers reported the intercellular transfer of functional P-gp protein from P-gp positive cells to P-gp negative cells both in vitro and in vivo (Levchenko et al., P.N.A.S. USA 102:1933-8, 2005). The transfer occurred between different cell types, and allowed the recipient drug-sensitive cells to survive toxic drug concentrations, leading to increased drug resistance. This may explain how sensitive cells acquire drug resistance.

Heat shock proteins (Hsps) belong to the family of stress proteins, some of which are induced by a variety of cellular stresses (Lindquist, Annu. Rev. Biochem. 55:1151-91, 1986). Several major Hsps (Hsp110, Hsp90, Hsp70, and Hsp25) are found in mammalian cells and are named in accordance with their molecular weights (Calderwood et al., Trends Biochem. Sci. 31:164-72, 2006). The Hsp70 family includes 2 major proteins: a constitutively expressed, 73-kDa protein (Hsc70) and a stress-inducible, 72-kDa protein (Hsp70). A major role of Hsps resides in their ability to function as molecular chaperones. Hsp70 binds nascent polypeptide chains; assists protein transport into the mitochondria, endoplasmic reticulum, and nucleus; maintains proper folding of precursor proteins; and protects proteins from stress (Bukau and Horwich, Cell 92:351-66, 1998; Craig et al., Cell 78:365-72, 1994; Georgopoulos and Welch, Annu. Rev. Cell Biol. 9:601-34, 1993; McKay, Adv. Protein Chem. 44:67-98, 1993). Hsp70 binds to misfolded proteins, enabling the damaged proteins to refold into their native state (Hartl and Hayer-Hartl, Science 295:1852-8, 2002; McLellan and Frydman, Nat Cell Biol. 3:E51-3, 2001; Wickner et al., Science 286:1888-93, 1999). Hsp70 also plays an important role in the control of cell cycle and growth. Under normal conditions, inducible Hsp70 is expressed in proliferating cells during G1/S and S phases of the cell cycle (Helmbrecht et al., Cell Prolif. 33:341-65, 2000).

In normal non-transformed cells, the expression of Hsp70 is low and is stress-inducible (Volloch and Sherman, Oncogene 18:3648-51, 1999). However, Hsp70 is abundantly expressed in most cancer cells (Calder wood et al., Trends Biochem. Sci. 31:164-72, 2006; Volloch and Sherman, Oncogene 18:3648-51, 1999; Kim et al., J. Korean Med. Sci. 13:383-8, 1998; Park et al., Gynecol. Oncol. 74:53-60, 1999; Yano et al., Japan. J. Cancer Res. 87:908-15, 1996). Hsp70 has been shown to play an active role in oncogenic transformation, and turning off the Hsp70 expression was shown to reverse the transformed phenotype of fibroblasts (Jaattela, Int. J. Cancer 60:689-93, 1995; Seo et al., Biochem. Biophys. Res. Commun. 218:582-7, 1996). Overexpression or induced endogenous levels of Hsp70 potently inhibits apoptosis (Calderwood et al., Trends Biochem. Sci. 31:164-72, 2006; Demidenko et al., Cell Death Differ. 2005; Takayama et al., Oncogene 22:9041-7, 2003). Expression of inducible Hsp70 enhances the proliferation of breast cancer cells in vitro (Barnes et al., Cell Stress Chap. 6:316-25, 2001). Furthermore, expression of Hsp70 correlates with increased cell proliferation, poor differentiation, lymph node metastases, and poor therapeutic outcome in human breast cancer (Ciocca et al., J. Natl Cancer Inst. 85:570-4, 1993; Lazaris et al., Breast Cancer Res. Treat. 43:43-51, 1997; Vargas-Roig et al., Cancer Detect. Prev. 21:441-51, 1997; Vargas-Roig et al., Int. J. Cancer 79:468-75, 1998). Hsp70 inhibits the mitochondrial pathway of apoptosis by blocking Apaf-1—mediated activation of caspase-9 and -3, as well as by repressing the activity of caspase-3 (Beere et al., Nat. Cell Biol. 2:469-75, 2000; Gabai et al., Mol. Cell. Biol. 22:3415-24, 2002; Jaattela et al., Embo. J. 17:6124-34, 1998; Saleh et al., Nat. Cell Biol. 2:476-83, 2000). Additionally, Hsp70 can also inhibit caspase-independent apoptosis by directly interacting with apoptosis-inducing factor (AIF), thereby preventing nuclear import and DNA fragmentation by AIF (Gurbuxani et al., Oncogene 22:6669-78, 2003; Ravagnan et al., Nat. Cell Biol. 3:839-43, 2001). Further, Hsp70 was shown to inhibit apoptosis signaling upstream to mitochondria by inhibiting Bax conformational change and localization to mitochondria. Also, by up-regulating STAT5 levels and activity, Hsp70 induces Bcl-xL and Pim-2 levels, thereby augmenting resistance to apoptosis exerted at the level of the mitochondria (Guo et al., Blood 105:1246-55, 2005). Studies show that Hsp70 contributes to Bcr-Abl-mediated resistance to apoptosis due to antileukemia agents such as Ara-C and etoposide (Guo et al., Blood 105:1246-55, 2005) and abrogation of Hsp70 can sensitize leukemia cells to therapy (Guo et al., Cancer Res. 65:10536-44, 2005). Other studies in breast and prostate cancer cells show that the inhibition of Hsp70 synthesis in tumor cells sensitizes them to chemotherapy (Jaattela et al., Embo. J. 17:6124-34, 1998; Gabai et al., Oncogene 24:3328-38, 2005; Kaur et al., Int. J. Cancer 85:1-5, 2000; Wei et al., Cancer Immunol. 40:73-8, 1995). Thus, downregulation of Hsp70 has been suggested as a potential approach to overcome tumor drug-resistance (Nylandsted et al., P.N.A.S. USA 97:7871-6, 2000).

Initially named as post-transcriptional gene silencing, RNA interference (RNAi) occurs in a variety of organisms (Meister and Tuschl, Nature 431:343-9, 2004). It is triggered by long double-stranded RNAs (dsRNAs) that could vary in length and origin. Upon introduction, the long dsRNAs enter a cellular pathway that is commonly referred to as the RNAi pathway. First, the dsRNAs get processed into 20-25 nucleotide siRNAs by an RNase III-like enzyme called Dicer. The siRNAs assemble into endoribonuclease-containing complexes known as RNA-induced silencing complexes (RISCs), unwinding in the process. The siRNA strands guide the RISCs to complementary RNA molecules, where they cleave and destroy the cognate RNA. Several groups independently reported that Argonaute2 protein is the “Slicer”, the enzyme that cleaves the mRNA (Meister and Tuschl, Nature 431:343-9, 2004; Rand et al., P.N.A.S. USA 101:14385-89; Liu et al., Science 305: 1437-41, 2004; Song et al., Science 305:1434-7, 2004). In mammalian cells, introduction of dsRNAs (>30 nucleotides) initiates a potent antiviral response, resulting in nonspecific inhibition of protein synthesis and RNA degradation (Williams, Biochem. Soc. Trans. 25:509-13, 1997). In 2001, Elbashir and others proposed the use of siRNA duplexes of 21-neucleotide length for RNA interference (Elbashir et al., Nature 411:494-8, 2001) to overcome antiviral response. While some studies have raised concerns over the possibility of siRNAs eliciting immune reactions via interactions with Toll-like receptor 3 and consequent interferon responses (Kim et al., Nat. Biotechnol. 22:321-5, 2004; Bridge et al., Nat. Genet. 34:263-4, 2003; Sledz et al., Nat. Cell Biol. 5:834-9, 2003), other studies have shown that it is possible to administer synthetic siRNAs to mice and downregulate an endogenous target without inducing interferon response (Heidel et al., Nat. Biotechnol. 22:1579-82, 2004).

Previous studies have shown the efficacy of siRNA-mediated P-gp gene silencing in overcoming drug resistance (Pichler et al., Clin. Cancer Res. 11:4487-4494, 2005; Xu et al., Mol. Ther. 11:523-530, 2005; Xu et al., J. Pharmacol. Exp. Ther. 302:963-71, 2002; Yague et al., Gene Ther. 11:1170-4, 2004; Zhang et al., Gynecol. Oncol. 97:501-507, 2005). These studies demonstrate that inhibition of P-gp expression by siRNA enhances intracellular accumulation of P-gp substrates and sensitizes resistant cells to anticancer agents. Stable transfection of a siRNA to Hsp70 in human acute myelogenous leukemia HL-60 cells (HL-60/Hsp70) and in Bcr-Abl-expressing cultured CML-BC K562 cells completely abrogated the endogenous levels of Hsp70 and blocked 17-allylamino-demethoxy geldanamycin-mediated Hsp70 induction, sensitizing cells to drug-induced apoptosis (Guo et al., Blood 105:1246-55, 2005). Similarly, siRNA-mediated knockdown of Hsp70 expression in K562 cells induced marked sensitivity to paclitaxel-induced apoptosis (Ray et al., J. Biol. Chem. 279:35604-15, 2004). However, a major obstacle to the use of siRNA for clinical therapy is the transient nature of gene silencing observed with conventional siRNA delivery methods. This is due to the rapid degradation of siRNA in plasma and cellular cytoplasm, resulting in its short half-life. Thus, in the study by Xu et al, in which Lipofectamine® was used for transfecting cells with siRNA, inhibition of gene expression was achieved for only 2-3 days. Similarly, a transient (<48 hrs) inhibition was observed when Oligofectamine® was used for transfection (Wu et al., Cancer Res. 63: 1515-9, 2003). As the Examples indicate, sustained inhibition of the protein activity is essential for sustaining the cytotoxicity of paclitaxel in resistant cells. Viral vectors produce stable inhibition of gene expression (Pichler et al., Clin. Cancer Res. 11:4487-4494, 2005; Xu et al., Mol. Ther. 11:523-530, 2005); however, viral vectors are associated with concerns of toxicity and immunogenicity (Merdan et al., Adv. Drug Deliv. Rev. 54:715-58, 2002; Schagen et al., Crit. Rev. Oncol. Hematol. 50:51-70, 2004;). Another issue that needs to be considered when using gene silencing to overcome drug resistance is the potential for kinetic differences in gene silencing and drug's availability at the target site. For optimum efficacy, the drug should be available in the tumor cell when the gene is silenced. This forms the rationale for formulating siRNA and drug in the same formulation, which will ensure that both siRNA and drug are presented to the tumor cell at the same time.

### Nanoparticles

Nanoparticles of various polymers may be used with certain embodiments disclosed herein. Preferable polymers include hydrophobic polymers, and even more preferably biodegradable, bioresorbable, or bioerodable polymers. Non-limiting examples of polymers that are considered to be biodegradable, bioresorbable, or bioerodable include, but are not limited to, aliphatic polyesters; poly(glycolic acid) and/or copolymers thereof (e.g., poly(glycolide trimethylene carbonate); poly(caprolactone glycolide); poly(lactic acid) and/or isomers thereof (e.g., poly-L(lactic acid) and/or poly-D (lactic acid) and/or copolymers thereof (e.g. DL-PLA), with and without additives (e.g. calcium phosphate glass), and/or other copolymers (e.g. poly(caprolactone lactide), poly(lactide glycolide), poly(lactic acid ethylene glycol); poly(ethylene glycol) (in its various weights, i.e. 2000 D, 4000 D, 6000 D, 8000 D, etc.); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that considered to be biostable include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g. polystyrene); poly(vinyl ethers) (e.g. polyvinyl methyl ether); poly(vinyl ketones); poly(vinylidene halides) (e.g. polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g. polyvinyl chloride); polyacrylic acid; ethylene acrylic acid copolymer; ethylene vinyl acetate copolymer; polyvinyl alcohol; poly(hydroxyl alkylmethacrylate); Polyvinyl esters (e.g. polyvinyl acetate); and/or copolymers, blends, and/or composites of above. Non-limiting examples of polymers that can be made to be biodegradable and/or bioresorbable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used.

Examples of some preferred polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of natural polymers that may be utilized herein include proteins such as albumin, collagen, gelatin and prolamines, for example, zein, and polysaccharides such as alginate, cellulose derivatives and polyhydroxyalkanoates, for example, polyhydroxybutyrate.

In certain embodiments, the nanoparticles disclosed herein can be of any particular size, depending on the goal of the embodiment (therapeutic agent release, tissue or blood vessel penetration, toxicity, bioavailability, etc.). In certain embodiments, the nanoparticle size is in the range of about 5 nm to about 10,000 nm or any value there between or less, or greater. In certain embodiments, the nanoparticle size is about 5 nm, about 10 nm, about 20 nm, about 30 nm, about 40 nm, about 50 nm, about 60 nm, about 70 nm, about 80 nm, about 90 nm, about 100 nm, about 200 nm, about 300 nm, about 400 nm, about 500 nm, about 600 nm, about 700 nm, about 800 nm, about 900 nm, about 1,000 nm, about 2,000 nm, about 2,500 nm, about 3,000 nm, about 3,500 nm, about 4,000 nm, about 4,500 nm, about 5,000 nm, about 5,500 nm, about 6,000 nm, about 6,500 nm, about 7,000 nm, about 7,500 nm, about 8,000 nm, about 8,500 nm, about 9,000 nm, about 9,500 nm, about 10,000 nm, or any value there between or greater.

Nanoparticles formulated using a FDA-approved, biodegradable polymer PLGA are used in the disclosed studies. The inventors' previous studies have demonstrated that PLGA nanoparticles are non-toxic and biocompatible (1), and are suitable for in vivo drug delivery (Panyam et al., J. Drug Target. 10:515-23, 2002). We have previously shown that nanoparticles can efficiently encapsulate and sustain the release of hydrophobic drugs like dexamethasone (Panyam et al., J. Pharm. Sci. 93:1804-14, 2004) and paclitaxel and nucleic acids (Prabha et al., Int. J. Pharm. 244:105-15, 2002). An important advantage of PLGA nanoparticles is that the rate of drug/nucleic acid release from nanoparticles, and therefore, the therapeutic efficacy, can be controlled by varying the polymer properties such as molecular weight, lactide-glycolide ratio and end-group chemistry (Panyam and Labhasetwar, Mol. Pharm. 1:77-84, 2004; Prabha and Labhasetwar, Pharm. Res. 21:354-64, 2004).

The inventors' previous studies have shown that PLGA nanoparticles are taken up rapidly by cells by endocytosis, resulting in higher cellular uptake of the entrapped therapeutic agent (Panyam and Labhasetwar, Pharm. Res. 20:212-20, 2003). Mechanistic studies have shown that both clathrin-coated pit endocytosis and fluid-phase pinocytosis are involved. Following their uptake, nanoparticles enter the endo-lysosomal pathway, and are localized in both primary/recycling endosomes and in secondary endosomes and lysosomes. Nanoparticles escape the endo-lysosomal pathway into the cytoplasm through a process of surface charge reversal. The surface charge of nanoparticles changes from anionic to cationic in the acidic pH of secondary endosomes/lysosomes, because of migration of protons from the bulk liquid to the nanoparticle surface. Surface charge reversal results in the interaction of nanoparticles with the anionic lysosomal membrane, leading to the escape of nanoparticles into the cytoplasm (Panyam et al., Faseb J 16:1217-26, 2002). Following entry, nanoparticles are retained in the cytoplasm for a sustained period of time (1). Thus, nanoparticles act as intracellular drug/gene depots, slowly releasing the encapsulated therapeutic agent in the cellular cytoplasm. This results in enhanced therapeutic efficacy for drugs like dexamethasone (Panyam and Labhasetwar, Mol. Pharm. 1:77-84, 2004) and paclitaxel, because cytoplasm is the site of action for these drugs.

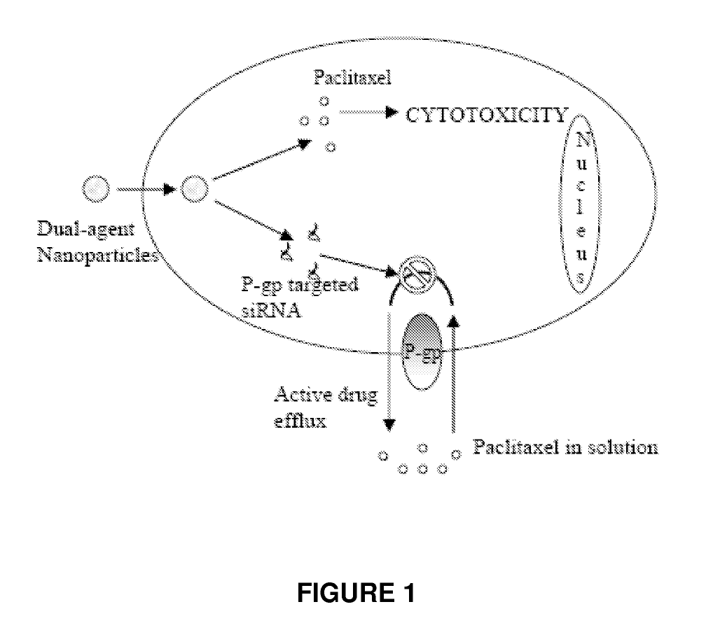

The proposed mechanism of action of dual-agent nanoparticles is represented in FIG. 1. Following their uptake and endolysosomal Paclitaxel escape, nanoparticles are expected cytotoxicity to sustain the cytoplasmic release of both siRNA and paclitaxel, resulting in the inhibition of target protein (P-gp or Hsp70) expression and reversal of resistance to paclitaxel. The therapeutic efficacy of nanoparticles is further enhanced by their ability to protect both drug and siRNA from degradation by lysosomal enzymes (Prabha and Labhasetwar, Mol. Pharm. 1:211-219, 2004). Nanoparticles, because of their colloidal nature and serum stability (Panyam and Labhasetwar, Pharm. Res. 20:212-20, 2003), can be easily dispersed in saline and injected intravenously. According to this disclosure, nanoparticle-encapsulated paclitaxel is susceptible to P-gp-mediated drug efflux, and inhibition of P-gp reverses resistance to nanoparticle-encapsulated paclitaxel.

### Therapeutic or Active Agents

Certain embodiments disclosed herein relate to compositions and methods relating to treating at least one therapeutic condition and/or diseases with the compositions made by the disclosed methods. As used herein, “treat,” “treatment,” “treating,” and all derivations thereof may refer to preventing or ameliorating at least one symptom of a disease or condition in a subject in need thereof, such as a mammal, and preferably a human. In certain embodiments, at least one condition or disease is related to a pulmonary condition or disease. In other particular embodiments, at least one condition or disease is related to a systemic condition or disease. In other particular embodiments, at least one condition or disease is related to a local condition or disease. In other particular embodiments, the compositions and/or methods described herein relate to delivery of preventative drug formulations, including cytotoxic anti-tumor agents.

In certain embodiments, the nanoparticles described herein further comprise at least one therapeutic and/or active agent joined thereto. Various therapeutic or active agents can be utilized with the nanoparticles, depending on the desired diagnostic and/or therapeutic outcome. For example, ligands and/or antibodies can be selected based on receptor expression of tumor and/or tissue specificity, and joined to the nanoparticles described herein. In certain embodiments, active agents may be selected to induce cell proliferation (e.g. for wound or blood vessel repair), to directly or indirectly cause necrosis or apoptosis (e.g. for tumor destruction or for microbial infection), or to induce cell differentiation (e.g. for wound repair).

Some examples of therapeutic or active agents that may be utilized with the instant disclosure include but are not limited to: polysaccharides, steroids, analgesics, anti-inflammatory agents, antimicrobial agents, anti-malarial agents, hormonal agents including contraceptives, amino acids, peptides, polypeptides, proteins, glycoproteins, other chemically or biologically modified proteins, anti-neoplastic agents, angiogenic agents, anti-angiogenic agents, photosensitizers, cytokines, cytokine receptors, enzymes, fats, vaccines and diagnostic agents.

Therapeutic or active agents may further comprise nucleic acids, present as bare nucleic acid molecules, viral vectors, associated viral particles, nucleic acids associated or incorporated within lipids or a lipid-containing material, plasmid DNA or RNA or other nucleic acid construction of a type suitable for transfection or transformation of cells. In certain embodiments, the active agent comprises a small molecular weight pharmaceutical drug. In other embodiments, the active agent comprises at least one large biomolecule, including but not limited to peptides, polypeptides, proteins, amino acids (including naturally occurring as well as non-natural amino acids or amino acid analogues), nucleotides, DNA, RNA, tRNA, mRNA, rRNA, shRNA, microRNA, and any combinations thereof, or the like. The active agents may be in various forms, such as soluble and insoluble charged or uncharged molecules, components of molecular complexes or pharmacologically acceptable salts.

In certain embodiments, the active agent comprises folic acid, or RGD (Arg-Gly-Asp) peptide.

Folic acid as a ligand is disclosed herein for tumor-targeted drug delivery. Folate receptor is overexpressed on many human cancer cell surfaces (Turk et al., Arthritis Rheum. 46:1947-55, 2002). Although the reduced folate carrier is present in virtually all cells, folate-conjugates are not substrates and are taken up only by cells expressing functional folate receptors (Hilgenbrink and Low, J. Pharm. Sci. 94:2135-46, 2005). Folic acid conjugation allows endocytic uptake of the conjugated carrier via the folate receptor, resulting in higher cellular uptake of the encapsulated drug (Mansouri et al., J. Biol. Chem. 269:3198, 1994). The high affinity of folic acid to its receptor (binding constant ˜1 nm) allows its use for specific cell targeting. The ability of folic acid to bind its receptor is not altered by covalent conjugation to delivery systems. A novel approach to incorporate folic acid on nanoparticles is described in detail in the Examples.

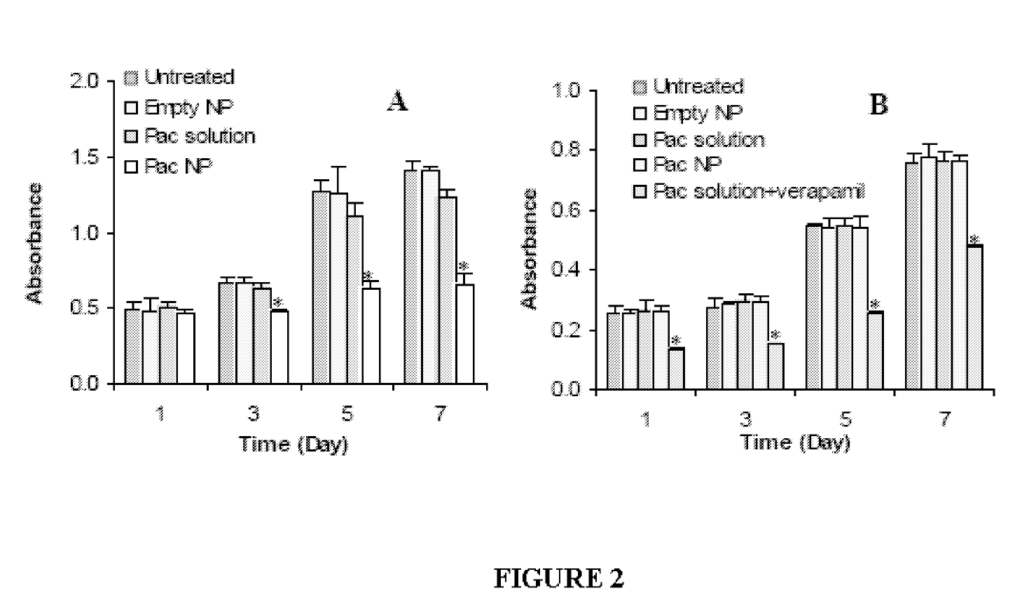

Nanoparticle-encapsulated paclitaxel is cytotoxic to drug-sensitive but not resistant cells. The inventors' previous studies have shown that nanoparticles, following endo-lysosomal escape, deliver the encapsulated drug into the cytoplasm (Panyam and Labhasetwar, Mol. Pharm. 1:77-84, 2004). It was to determine that paclitaxel delivered into cellular cytoplasm is susceptible to P-gp-mediated drug efflux, because the “vacuum cleaner” hypothesis suggests that P-gp extracts the drug as the drug diffuses into the cell through the lipid bi-layer. Hence, it was not known whether drug delivered into the cytoplasm can be effluxed by P-gp. The inventors initially investigated the efficacy of paclitaxel encapsulated in nanoparticles in drug-sensitive MCF-7 cells. At the concentration tested, paclitaxel in solution demonstrated a marginal but significant (P<0.05) inhibition of cell proliferation compared to untreated cells. However, significantly higher and more sustained (for up to 7 days) inhibition of cell proliferation was obtained when the cells were treated with paclitaxel-loaded nanoparticles (P<0.05 for nanoparticles and solution groups for all time points, FIG. 2A). The inventors investigated the efficacy of the same treatments in NCl/ADR-RES cells. These cells overexpress P-gp, and are resistant to paclitaxel. As can be seen in FIG. 2B, treatment with paclitaxel, in solution or in nanoparticles, had no significant effect on the viability of cells. Addition of 100 μM verapamil, a P-gp inhibitor, resulted in the reversal of drug resistance, confirming that drug resistance in this cell line was to due to P-gp. These studies suggest that nanoparticle-encapsulated paclitaxel is susceptible to P-gp-mediated drug resistance.

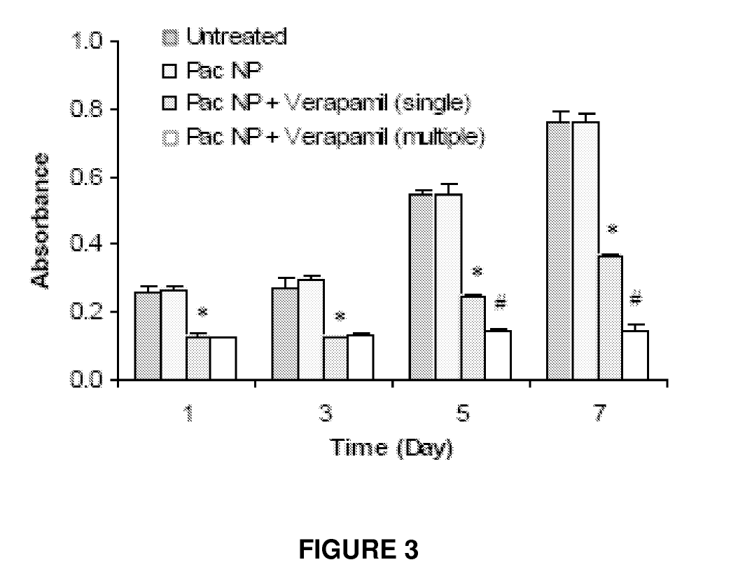

In order to verify that resistance to nanoparticle-encapsulated paclitaxel is due to P-gp activity, the inventors tested the effect of verapamil on the cytotoxicity confirming that resistance to nanoparticle-encapsulated paclitaxel is due to P-gp (FIG. 3). The inventors also studied the effect of transient Vs sustained inhibition of P-gp on cytotoxicity of nanoparticle-encapsulated paclitaxel. As FIG. 3 indicates, transient inhibition of P-gp resulted in only transient cytotoxicity of nanoparticle-encapsulated paclitaxel. However, sustained inhibition of P-gp by continuously incubating cells with verapamil resulted in sustained cytotoxicity with nanoparticle-encapsulated paclitaxel. These data suggest that sustained inhibition of P-gp is required for sustaining the cytotoxicity of nanoparticle-encapsulated paclitaxel in drug-resistant cells.

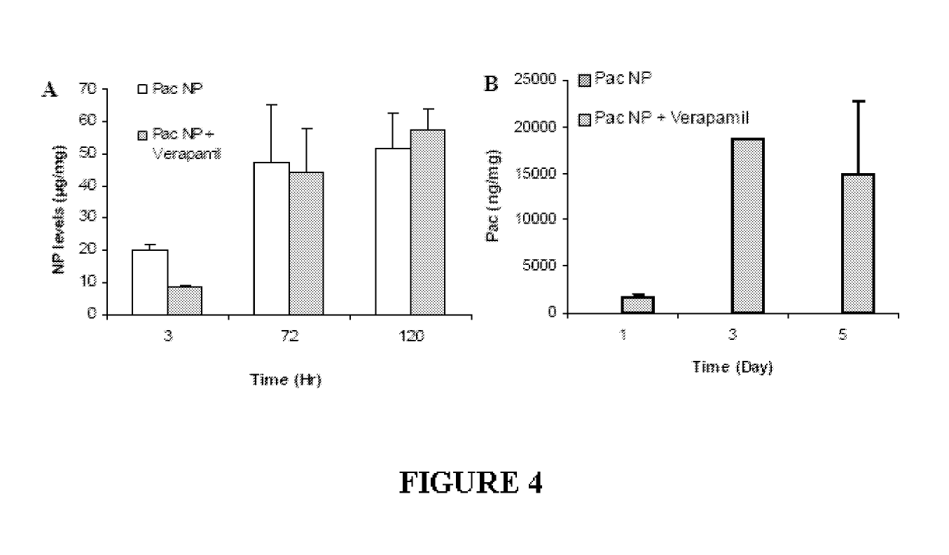

In order to verify that differences in drug accumulation are not due to differences in nanoparticle uptake/retention in cells, the inventors labeled nanoparticles with 6-coumarin, and followed the cell uptake and retention of nanoparticles and paclitaxel in NCl/ADR-RES cells. 6-Coumarin is a highly lipophilic dye that has been previously used as a marker for nanoparticles in cell uptake studies (Panyam et al., Faseb J. 16:1217-26, 2002). As can be seen in FIG. 4, inhibition of P-gp by verapamil did not significantly increase the uptake or retention of nanoparticles. However, cellular accumulation of nanoparticle-encapsulated paclitaxel was significantly decreased by P-gp activity, suggesting that P-gp does not affect uptake or retention of nanoparticles but decreases the accumulation of nanoparticle-encapsulated paclitaxel.

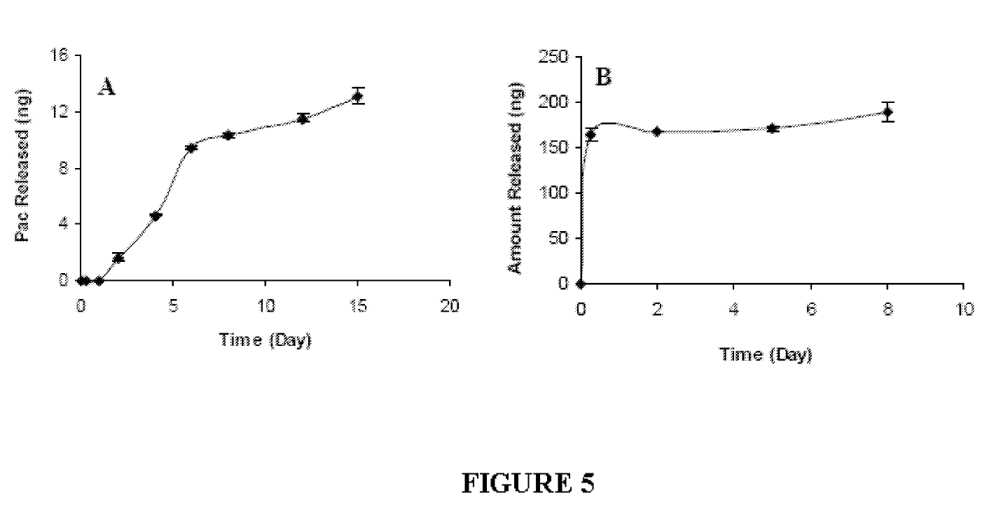

One objective of the Examples herein was to investigate the release of P-gp-targeted siRNA and paclitaxel from nanoparticles in phosphate buffered saline. As can be seen in FIG. 5, nanoparticles sustained the release of both siRNA and paclitaxel. The release of siRNA was similar to that observed for other macromolecules like plasmid DNA and protein (Prabha et al., Int. J. Pharm. 244:105-15, 2002; Panyam et al., J. Control Release 92:173-87, 2003), with an initial burst release followed by a lag-phase. Nanoparticles released paclitaxel with an initial lag phase (24 hrs), followed by a more continuous release. Nanoparticles (8 μg) released a total of 108 ng of paclitaxel over 15 days (release rate≈7 ng/day/8 μg).

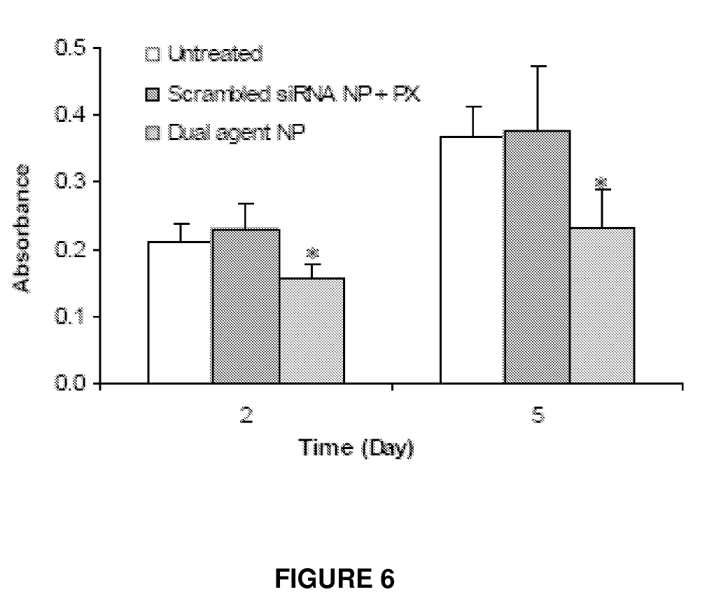

In certain embodiments, multiple therapeutic or active agents may be utilized. The efficacy of dual-agent nanoparticles in overcoming tumor drug resistance was investigated. NCl/ADR-RES cells were treated with a single-dose of dual-agent nanoparticles releasing 7 ng/day/8 μg paclitaxel and 0.3 ng/day/8 μg siRNA. The doses of siRNA and paclitaxel were derived from studies with nanoparticles containing only siRNA and nanoparticles containing only paclitaxel (data not shown). As can be seen in FIG. 6, dual-agent nanoparticles resulted in significant (P<0.05) cytotoxicity in NCl/ADR-RES cells compared to controls. Cytotoxicity was sustained for up to 5 days, suggesting sustained P-gp inhibition. Treatment with non-targeted siRNA nanoparticles and paclitaxel did not have any effect on cell viability, confirming that observed cytotoxicity is not due to non-specific gene inhibition. The inventors expect that greater and more sustained cytotoxicity can be demonstrated by further optimizing siRNA and paclitaxel release rates from dual-agent nanoparticles.

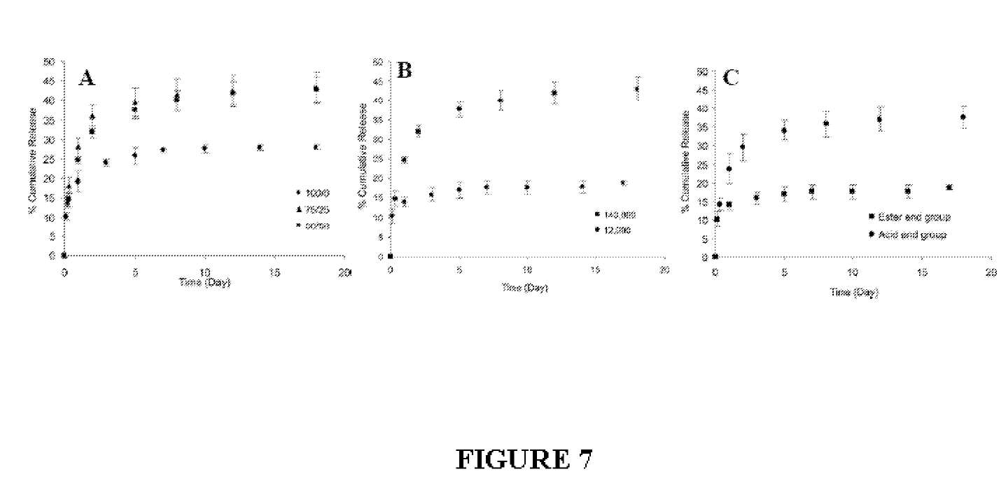

Nanoparticle formulations with different drug release rates (FIG. 7) were obtained by formulating nanoparticles with polymers of different compositions and molecular weights. Dexamethasone was used as a model hydrophobic drug. In vitro release of the drug from nanoparticles was found to be dependent on the lactide-to-glycolide ratio, molecular weight of the polymer and the end-group chemistry. Thus, nanoparticles formulated from 100% lactide content released lower percent of the encapsulated drug than those prepared from polymers containing glycolide (FIG. 7A). Nanoparticles formulated using low molecular weight polymer showed lower percent cumulative release (FIG. 7B). Also, lower cumulative percent drug release was obtained from nanoparticles prepared using polymers containing ester-end groups than from nanoparticles prepared using polymers containing acid end groups (FIG. 7C). These studies demonstrate that rate and extent of drug release from PLGA nanoparticles can be controlled by varying the properties of the polymer used. (Panyam et al., J. Pharm. Sci. 93:1804-14, 2004)

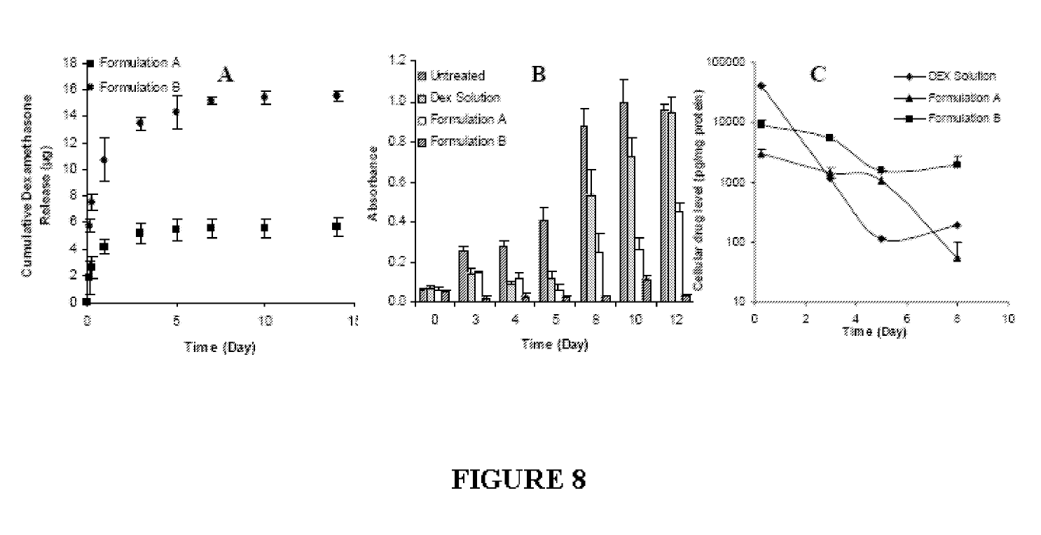

The present disclosure also demonstrates the relationship between the dose of the drug released and therapeutic efficacy. Dexamethasone, a lipophilic drug with cytoplasmic site of action, was used as a model drug. Two formulations with different release rates were selected for the studies. Formulation A (600 μg of nanoparticles) released a total of 6 μg of dexamethasone over 14 days, while the same amount of formulation B released a total of 16 μg over 14 days (FIG. 8A). Formulation A had a lower drug loading 5.6% (w/w) and 30% entrapment efficiency than formulation B 9.5% (w/w) and 46% entrapment efficiency. The two formulations were compared with drug in solution for their in vitro cytotoxicity. Treatment of cells with drug in solution demonstrated transient cytotoxicity compared to untreated cells (FIG. 8B). Cytotoxicity was seen up to 5 days following treatment; however, the level of cell proliferation increased beyond this point, and there was no significant difference in cytotoxicity between the untreated and treated cells on day 12.

Significantly higher and more sustained (for up to 12 days) cytotoxicity was obtained when the cells were treated with drug-loaded nanoparticles (p<0.05 for formulation B and solution groups for all time points and p<0.05 for formulation A and the solution group from day 8 to day 12). Within the two nanoparticle formulations, nanoparticles exhibiting a smaller amount of drug release (formulation A) produced a lower level of inhibition of cell proliferation compared to those with which exhibited a higher level of drug release (formulation B) (p<0.05 after day 5). Duration and extent of cytotoxicity correlated with the cellular drug accumulation. As can be seen in FIG. 8C, dexamethasone solution resulted in transient drug levels; formulation B resulted in sustained and significantly higher drug levels than formulation A, which released smaller dose of drug. These studies demonstrate that efficacy of nanoparticle-encapsulated drug depends on the dose of the drug released. (Panyam and Labhasetwar, Mol. Pharm. 1:77-84, 2004)

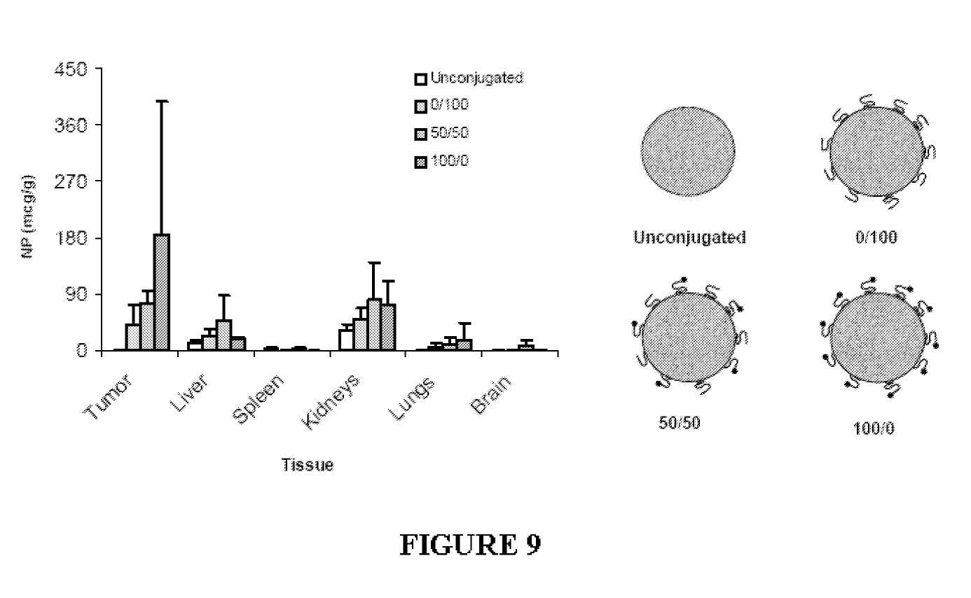

Another objective of the disclosure was to determine the effect of folic-acid conjugation on nanoparticle accumulation in target tumor tissue. Drug-resistant JC (murine breast adenocarcinoma) tumor xenografts were used. Nanoparticles were prepared by emulsion-solvent evaporation technique and PEG and PEG/folic acid were introduced in nanoparticles using a novel technique developed in the inventors' laboratory. Nanoparticles were labeled with 6-coumarin, a lipophilic fluorescent dye, for biodistribution studies. Nanoparticles containing PEG-folate and PEG in different ratios were injected intravenously through the tail vein. As can be seen from FIG. 9, nanoparticles without PEG and folic acid did not accumulate significantly in the tumor tissue. Addition of PEG significantly (p<0.05) increased tumor accumulation, and this effect was enhanced even more with the introduction of folic acid. These studies provide evidence for the ability of PEG and folic acid to enhance tumor targeting of nanoparticles.

In summary, the data disclosed herein demonstrate that dual-agent nanoparticles can overcome drug resistance and can be targeted to tumor cells using folic acid. These data support the conclusion that dual-agent nanoparticles will sustain the cellular delivery of siRNA and paclitaxel, resulting in enhanced paclitaxel accumulation and cytotoxicity, and ultimately, regression of resistant tumor.

### Functional Groups

As described for particular embodiments, the nanoparticles and methods of making the same may optionally include joining at least one functional group to the nanoparticle as well. Various functional groups may be utilized, depending on the desired outcome. For example, some non-limiting functional groups include hydrocarbons (containing an alkane, alkene, alkyne, benzene derivative, or toluene derivative); halogen containing groups (haloalkane, fluoroalkane, chloroalkane, bromoalkane, iodoalkane); oxygen containing groups (acyl halide, ketone alcohol, aldehyde, carbonate, carboxylate, carboxylic acid, ether, ester, hydroperoxide, peroxide); groups containing nitrogen (amide, amine, imide (such as maleimide), azide, azo compound imine, cyanate, isocyanate, nitrate, nitrile, nitrite, nitro compound, nitroso compound, pyridine derivative); groups containing phosphorus and sulfur (phosphine, phosphodiester, phosphonic acid, phosphate, sulfide or thioether, sulfone, sulfonic acid, sulfoxide, thiol, thiocyanate, disulfide) urea, urethane (carbamate), pyridine, indole, carbonate, thioester, arcylate/acrylic, amidine, ethyl, acid versions of aliphatic compounds that contain alkenes, alkanes or alkynes, imidazole, oxazole, and others. Each of these terms has its standard definition known to one skilled in the art.

### Detection Agents

In addition to the agents previously set forth, the nanoparticles and methods of making the same described herein may further comprise joining at least one detection agent to the nanoparticle. Detection agents may include any agent that is able to be quantitatively or qualitatively observed or detected. For example, a detection agent may be a fluorophore for imaging detection, a radio-isotope for radiographic detection, magnetic or paramagnetic agents for magnetic detection, an enzyme for enzymatic detection, and the like.

Some examples of detection agents include but are not limited to: biotin, streptavidin, green fluorescent protein (GFP), fluorescein (FITC), phycoerythrin (PE), Texas Red, ³²P, ³⁵S, ¹²⁵I, ³H, and others. In certain embodiments, the detection agent is detectable due to its inherent properties, and in other embodiments, the detection agent is detectable only upon induction with an inducing element (which may be a biological, chemical or physical element).

It should be understood that the Examples and embodiments described herein are for illustrative purposes only and that various modifications or changes in light thereof will be suggested to persons skilled in the art and are to be included within the spirit and purview of this application.

### EXAMPLES

### Example 1

### Dual-Agent Nanoparticles that Demonstrate Sustained Cytotoxicity

For sustained cytotoxicity, it is important that cytotoxic drug levels are maintained for a sustained period of time (Panyam and Labhasetwar, Mol. Pharm. 1:77-84, 2004). The premise for the present Example is that the duration of cytotoxicity of dual-agent nanoparticles depends on the rate of siRNA and paclitaxel release from nanoparticles. This Example entails the determination of cytotoxicity following treatment of drug-resistant tumor cells with nanoparticle formulations that release different doses of siRNA and paclitaxel. The results will be used to identify an optimal nanoparticle formulation that demonstrates sustained cytotoxicity (over 15 days) in resistant tumor cells.

Duration of 15 days is chosen based on the fact that this is the maximum duration over which cytotoxicity can be studied in vitro in different drug-sensitive and resistant cell lines. This Example yields data regarding the effect of dose of siRNA and paclitaxel on the cytotoxicity of dual-agent nanoparticles, and establishes sustained siRNA and paclitaxel delivery as the mechanism responsible for the efficacy of dual-agent nanoparticles. These data enable use of the optimized formulation in subsequent studies.

Cell lines. A panel of paclitaxel-resistant (P-gp or Hsp70 over-expressing) and sensitive cells will be used. MCF-7/Dox (breast) and Kbv (oral carcinoma) cells over-express P-gp. K562 (leukemia) and MCF7/Hsp70 cells over-express Hsp70. Kb, MCF-7 and HL-60 cells are sensitive to paclitaxel, and will be used as controls to make comparisons between resistant and sensitive cells. All the cell lines will be maintained and cultured as per published protocols.

Dual-agent nanoparticles that release different doses of siRNA and paclitaxel. The objective of the study is to formulate nanoparticles that release ˜5, 10, or 20 μg siRNA and ˜100, 200 or 400 μg paclitaxel (from ˜8 mg nanoparticles) over a 30-day period. These rates were chosen based on the fact that nanoparticles which released siRNA at the rate of ˜0.3 ng/day/8 μg nanoparticles and paclitaxel at the rate of ˜7 ng/day/8 μg nanoparticles were effective in drug-resistant tumor cells in vitro (see Preliminary Studies). Based on this, release of 0.3×30×1000≈10 μg siRNA and 7×30×1000≈200 μg paclitaxel from 8 mg nanoparticles were selected as median release rates.

Dual-agent nanoparticles will be formulated using a modification of the inventors' previously published double-emulsion solvent evaporation technique (90). In a typical procedure, siRNA solution in tris-EDTA buffer (0.2 ml) containing 2 mg bovine serum albumin is emulsified in PLGA solution (30 mg in 1 ml chloroform) containing paclitaxel by sonication using a probe sonicator (Misonix) to form a primary water-in-oil emulsion. The primary emulsion is further emulsified into 12 ml of aqueous 2% w/v polyvinyl alcohol solution by sonication. Precaution is taken to maintain the temperature of the emulsion around 4° C. during sonication in order to maintain the stability of siRNA. The emulsion is stirred overnight to evaporate chloroform.

Nanoparticles formed are recovered by ultracentrifugation (140,000×g), washed two times with nuclease-free water to remove unentrapped drug and siRNA, and then lyophilized for 48 hrs. To determine siRNA loading in nanoparticles, washings from the above formulation steps will be analyzed for siRNA concentration by Picogreen assay (Molecular Probes) to determine the quantity of siRNA that is not entrapped in nanoparticles. From the total amount of siRNA that was added in the formulation and the amount that is not entrapped in nanoparticles, siRNA encapsulated in nanoparticles will be determined.

To determine paclitaxel loading, nanoparticles will be incubated with methanol for 48 hrs, and the drug concentration in methanol extract will be determined by HPLC. A Shimadzu HPLC system consisting of Curosil-B column (Phenomenex) with UV detection (228 nm) will be used for drug quantification. Mobile phase consisting of ammonium acetate (10 mM, pH 4.0) and acetonitrile in the ratio of 55:45 v/v will be used at a flow rate of 1.0 ml/min. To determine in vitro release of siRNA, nanoparticles (1 mg/ml) will be suspended in sterile, nuclease free PBS (pH 7.4; 0.15 M), and kept at 37° C. and 100 rpm. At different time points, supernatants from release samples will be analyzed for siRNA by Picogreen assay.

To determine paclitaxel release, nanoparticles (1 mg/ml) will be suspended in PBS (pH 7.4; 0.15 M) containing 0.1% Tween 80 (to maintain sink conditions), and kept at 37° C. and 100 rpm. Paclitaxel concentration in the release buffer will be determined by HPLC. Nanoparticles that release different doses of siRNA and paclitaxel will be formulated by varying the dose-ratios of siRNA and paclitaxel in the formulation and by using polymers of different molecular weights and hydrophobicity. PLGA polymers of different molecular weights and composition are available commercially (Birmingham Polymers).

Nanoparticles with folic acid and PEG on the surface. Following the preparation of second emulsion in polyvinyl alcohol (see above), a methanol solution (100 μl) of polylactide (PLA)-PEG copolymer (1500-5000 Da) and/or PLA-PEG-folic acid conjugate (various ratios—100/0, 75/25, 50/50, 25/75, 0/100) is added to the emulsion. This results in the anchoring of the PLA segments into nanoparticles, with PEG and PEG-folic acid chains on the surface (FIG. 9). Following this, the emulsion is stirred to evaporate organic solvents and nanoparticles are processed as described above. This procedure was used to obtain nanoparticles containing PEG and PEG-folic acid conjugate on the surface (FIG. 9).

Sustained cytotoxicity. An objective of the Example is to demonstrate sustained cytotoxicity (over 15 days) of dual-agent nanoparticles in drug-resistant cells in vitro. Drug sensitive and drug resistant cells will be seeded at a density of 5×10³ cells/well in 96-well plates, and treated with formulations that release different doses of siRNA and paclitaxel. Nanoparticles containing only paclitaxel or siRNA, paclitaxel and siRNA in solution, nanoparticles containing non-targeted siRNA and paclitaxel, and empty nanoparticles will be used as controls.

Cytotoxicity will be determined as a function of time using a standard MTS assay (CellTiter 96 A_{Queous}, Promega). The medium will be changed on day 2 and every other day thereafter, and no further dose of the treatment will be added. At different time points, the MTS assay reagent will be added to each well and incubated for 150 min, and the absorbance will be measured at 490 nm using a microplate reader (Biotek). The correlation between cytotoxicity and siRNA/paclitaxel release and the optimal release that sustains cytotoxicity in resistant cells over 15 days will be determined. Using dual-agent nanoparticles that released 0.3 ng/day/8 μg P-gp-targeted siRNA and 7 ng/day/8 μg paclitaxel, we were able to sustain the cytotoxicity of dual-agent nanoparticles for 5 days. By optimizing the release rates of siRNA and paclitaxel further, we expect to achieve cytotoxicity in resistant cells over 15 days.

Induction of apoptosis. Treatment with paclitaxel results in induction of apoptosis, but tumor cells overexpressing P-gp or Hsp70 are resistant (Gabai et al., Mol. Cell. Biol. 22:3415-24, 2002; Larsen, et al., Pharmacol. Ther. 85:217-29, 2000). Thus, it is important to establish that dual treatment approach induces apoptosis in resistant cells. This will provide advanced confirmation regarding the efficacy of dual-agent nanoparticles in drug resistance. Induction of apoptosis will be studied by determining phosphatidylserine exposure and plasma membrane stability. Cells grown in culture will be treated with nanoparticle formulation that demonstrated maximal cytotoxicity and the respective controls as described above.

Cells will be stained with a combination of 2 μl of Annexin V-FLUOS™ and 2 μl of propidium iodide (1 μg/ml final concentration) in 100 μl of incubation buffer 10 mM Hepes (pH 7.4)/140 mM NaCl/5 mM CaCl₂ for 10 min on ice. Cells (10⁵ per sample) will then be analyzed in a flow cytometer using appropriate software. Cells binding annexin but not stained by propidium iodide will be considered apoptotic, whereas cells with higher propidium iodide fluorescence with or without bound annexin will be considered to be post-apoptotic necrotic or simply necrotic. It is expected that treatment with nanoparticles will result in higher induction of apoptosis than that with control treatments.

At the end of the protocol set forth in this Example 1, an optimal rate of siRNA and paclitaxel release from dual-agent nanoparticles is identified for sustaining paclitaxel cytotoxicity in resistant tumor cells. Nanoparticles formulated using a PLGA polymer of 50/50 lactide to glycolide ratio and ˜170 kDa molecular weight demonstrated paclitaxel release rate of 7 ng/day/8 μg; for the same polymer, siRNA release was 0.3 ng/day/8 μg. Furthermore, polymers with high lactide content or low molecular weight result in nanoparticles that demonstrate higher loading and greater release of a hydrophobic drug (Panyam et al., J. Pharm. Sci. 93:1804-14, 2004). On the other hand, polymers with higher glycolide content result in nanoparticles that demonstrate greater release of nucleic acid-type therapeutic agents (Prabha and Labhasetwar, Pharm. Res. 21:354-64, 2004). Thus, by using polymers of different composition, it is expected that nanoparticles may be obtained with the different release rates of siRNA and paclitaxel. Similarly, cytotoxicity of nanoparticle-encapsulated drug correlated with the dose of the drug released; therefore, it is expected that a positive correlation may be obtained between the dose of siRNA and paclitaxel released and the duration of cytotoxicity of dual-agent nanoparticles in resistant tumor cells. Overall, these studies may be used to design formulations that demonstrate sustained cytotoxicity.

As an alternative experimental method, if sustained P-gp inhibition with synthesized siRNA cannot be achieved, hairpin siRNAs can be expressed from stably integrated plasmids, because this approach could provide sustained gene inhibition (Yague et al., Gene Ther. 11:1170-4, 2004).

### Example 2

### Kinetics of Tumor-Targeting with Dual-Agent Nanoparticles In Vivo

The objective of this Example is to determine the kinetics of tumor targeting in a mouse tumor xenograft model with nanoparticles that are optimized for sustained cytotoxicity in vitro. This Example is designed to test the hypothesis that the presence of PEG and folic acid on the surface of nanoparticles will enhance tumor-targeting of nanoparticles. The approach used to test this hypothesis will be determination of kinetics of nanoparticle accumulation in tumor tissue following treatment with nanoparticle formulations with different amounts of PEG and folic acid in a mouse xenograft tumor model. Data will be obtained regarding the kinetics of drug and siRNA accumulation in tumor, including the rate and extent of nanoparticle accumulation in tumor tissue. This will enable determination of the dose of nanoparticles required for sustained tumor regression with dual-agent nanoparticles. This will result in improved design of subsequent studies on the therapeutic efficacy of dual-agent nanoparticles in vivo.

Tumor model. MCF-7 cells will be used for induction of tumors. MCF-7 is the parenteral cell line for MCF/Dox and MCF-7/HSP70 cells. MCF-7/Dox cells overexpress P-gp (Lee et al., J. Control Release 103:405-18, 2005) while MCF-7/Hsp70 cells overexpress Hsp70 (Barnes et al., Cell Stress Chaperones 6:316-25, 2001). MCF-7 cells overexpress folate receptors, and are therefore good model cells for tumors overexpressing folate receptors. Ovariectomized female NCRNU-M mice (Taconic Farms), 6-8 weeks old, will be used. Mice will be maintained exclusively on folate deficient rodent chow. Cells (5×10⁶) will be injected in the subcutaneous space near the flank. Tumor growth will be facilitated by implanting sustained-release 0.7 mg estradiol pellets (Innovative Research of America) in the subcutaneous space between the shoulders. After palpable tumor growth, tumor volume will be determined using calipers measuring the length (L) and width (W) of the tumor. Tumor volume will be calculated using the equation: (L×W²)/2. When tumor sizes are between 100 mm³ and 400 mm³, animals will be injected with 4 mg/kg of different nanoparticle formulations (Table 1). Nanoparticles will be labeled with 6-coumarin, a fluorescent dye, for the biodistribution studies (Panyam et al., Int. J. Pharm. 262:1-11, 2003).

TABLE 1
Treatment groups for pharmacokinetics study

| | | | Number of |
|---|---|---|---|
| Experiment | Group | Treatment | animals |
| Kinetics of | 1 | Folate-PEG/PEG nanoparticles | 6 × 7 = 42 |
| Tumor | | (100/0) | |
| Targeting | 2 | Folate-PEG/PEG nanoparticles | 6 × 7 = 42 |
| | | (50/50) | |
| | 3 | Folate-PEG/PEG nanoparticles | 6 × 7 = 42 |
| | | (0/100) | |
| | 4 | Folate-PEG/PEG nanoparticles | 6 × 7 = 42 |
| | | (0/0) | |

Animals will be euthanized at 1 hr, 6 hrs, 12 hrs, 24 hrs, 3 days, 1 week, and 2 weeks following treatment administration, and tumors as well as other organs including heart, liver, spleen, lungs, kidneys and brain will be harvested. Six animals will be used for each time point. Tissue samples will be homogenized using a tissue homogenizer in 0.5 ml cell culture lysis reagent (Promega). The tissue homogenates will be lyophilized, and 6-coumarin will be extracted with 1 ml methanol. 6-Coumarin concentrations in the extracts will be determined by HPLC as described previously (Panyam et al., Int. J. Pharm. 262:1-11, 2003). Results will be presented as rate of change of nanoparticle concentration (μg per gram of tissue) in tumor and other tissues. Tumor concentration C(t)—time t curve will be used to calculate area under time curve (AUC) and area under the moment curve (AUMC). Mean Residence Time (MRT) in the tumor will be calculated using the following formula:

$$MRT = \frac{AUMC}{AUC}$$

AUC will be used as measure of the ability of nanoparticles to specifically accumulate in tumor tissue. MRT will be used to determine the duration of tumor residence of nanoparticles. Data will be compared using the non-parametric Mann-Whitney test. Differences will be considered significant at P<0.05. Based on the amount of nanoparticles accumulating in tumor tissue and drug and siRNA loading in nanoparticles, amount of siRNA and paclitaxel delivered to tumor tissue will be determined.

Sustained inhibition of P-gp expression. An objective of this Example is to determine the kinetics of gene inhibition with dual-agent nanoparticles that are optimized for tumor targeting (above study). P-gp is used as a model target for these studies. MCF/Dox cells are used instead of the parent MCF-7 cells. Tumor bearing mice will be treated with a single intravenous injection of dual-agent nanoparticles. A dose of 8 mg of nanoparticles corresponding to 10 μg siRNA and 200 μg paclitaxel released over 30 days will be used (this formulation will be tested for in vitro cytotoxicity in coordination with Example 1). This is the median dose of siRNA and paclitaxel that is used in the dose-response study in Example 3. Following treatment administration, animals will be euthanized, and tumors will be harvested at different time points (1, 7, 14, 30, 60 and 90 days). Tumors will be examined for P-gp expression by both immunoblot analysis and real-time RT-PCR as described below. Three animals will be used for each time point. Animals treated with nanoparticles containing only siRNA, nanoparticles containing non-targeted siRNA and paclitaxel, and siRNA and paclitaxel with a commercial transfection reagent (Oligofectamine®) will be used as controls (Table 2). P-gp expression will be compared with that in vehicle-treated tumors. siRNA-loaded nanoparticles are expected to result in sustained and significant inhibition of P-gp expression compared to the controls. Transfection with the commercial transfecting reagent is expected to result in only transient gene silencing as the effect is lost once the siRNA delivered in the cell is degraded (Wu et al., Cancer Res. 63:1515-9, 2003). This Example will help determine the time period for which dual-agent nanoparticles are capable of suppressing gene expression. The resulting data will be used to determine the dosing frequency in Example 3.

Immunoblot analysis: Tumors will be homogenized in 0.1 ml of ice-cold PBS, and the cellular proteins will be precipitated with 6% w/v trichloroacetic acid. The precipitated proteins in the tissue homogenates will be dissolved in Laemmli disaggregating buffer. Dissolved proteins will be resolved by 7.5% SDS-PAGE and then transferred to PVDF membranes. Immunoblots will be incubated with a 1:500 dilution of P-gp primary antibody (clone Ab-1, Oncogene Science), followed by a 1:2000 dilution of secondary antibody goat anti-rabbit IgG-HRP (Bio-Rad). Signals will be detected with chemiluminescence reagents (Amersham) followed by exposure to Hyperfilm-ECL (Amersham).

Quantitative real-time RT-PCR: Expression of P-gp mRNA transcripts in tumor cells will be determined by RT-PCR using thermal cycler and analysis software (Eppendorf). Total RNA from the tumor homogenates will be extracted using the RNeasy Mini kit (Qiagen, Valencia, Calif.) according to the manufacturer's instructions. Oligonucleotides for MDR1 gene (forward primer: 5′-CTGCTTGATGGCAAAGAAATAAAG-3′) (SEQ ID NO:1), (reverse primer: 5′-GGCTGTTGTCTCCATAGGCAAT-3′) (SEQ ID NO:2), and probe (5′-6-FAM-CAGTGGCTCCGAGCACACCTGG-BHQ1-Q) (SEQ ID NO:3) will be used according to previously published methods (Sampath et al., Mol. Cancer. Ther. 2:873-884, 2003). Oligonucleotide sequences for human β-actin (forward primer, 5′-TGCGTGACATTAAGGAGAAG) (SEQ ID NO:4), reverse primer (5′-GCTCGTAGCTCTTCTCCA) (SEQ ID NO:5) will be used as internal control. PCR products will be separated on a 1% agarose gel containing ethidium bromide. The DNA fragments will be visualized by Bio-Rad Gel Doc system. Relative fluorescence values of PCR product will be calculated using a standard curve consisting of 0.1-1000 ng of template cDNA during sample analysis. MDR1 cDNA levels will be normalized by processing the same cell samples in a parallel reaction for β-actin mRNA levels. Relative expression values will be calculated as defined by Pfaffl (Pfaffl, Nuc. Acids Res. 29:e45, 2001) and data will be normalized to β-actin.

TABLE 2
Treatment groups for gene expression study

| | | Number of |
|---|---|---|
| Group | Treatment | animals |
| 1 | Dual-agent nanoparticles with PEG/folate | 6 × 3 = 18 |
| 2 | Dual-agent (non-targeted siRNA) nanoparticles with | 6 × 3 = 18 |
| | PEG/folate | |
| 3 | Nanoparticles containing only siRNA with | 6 × 3 = 18 |
| | PEG/folate | |
| 4 | siRNA + Oligofectamine ® + Paclitaxel | 6 × 3 = 18 |
| 5 | Vehicle | 6 × 3 = 18 |

Folic acid enhances tumor accumulation of nanoparticles. Nanoparticles that target tumor tissue are expected to stay in tumor for a prolonged period of time because of enhanced permeation and retention effect (Koziara et al., J. Control Release 112:312-9, 2006). In vitro release studies indicate that nanoparticles release about 10% of the encapsulated siRNA over an 8-day period. Because the rate of release of macromolecules from PLGA nanoparticles decreases with time (diffusion-dependent kinetics), nanoparticles are expected to sustain the in vivo release of encapsulated siRNA and inhibition of P-gp expression over a 30-45 day period. According to published studies (Prabha et al, Pharm. Res. 21:354-64, 2004; Prabha dissertation, Pharm. Sci., Univ. NE Med. Cntr., pp. 205, 2004), PLGA nanoparticles that showed similar release kinetics of encapsulated plasmid DNA (10% release over a 7-day period) in vitro, demonstrated sustained (over 5 weeks) gene expression in vivo.

It is possible that a lag-time in gene silencing could be observed, because siRNAs act only after the mRNAs are synthesized. However, preliminary studies suggest that, despite the potential timing problem, dual-agent nanoparticles are able to overcome P-gp-mediated drug efflux in vitro. Because nanoparticles release the encapsulated drug with a 24-hr lag and both siRNA and paclitaxel are released over a period of days, a <24 hr delay in gene silencing is not expected to significantly affect the therapeutic efficacy of nanoparticles. Further, an optimal formulation that will synchronize gene silencing with drug delivery may be identified from the studies in Example 1.

### Example 3

### In Vivo Anti-Tumor Efficacy of Dual-Agent Nanoparticles

A number of delivery vectors that demonstrate good efficacy in vitro do not perform as well in vivo due to instability in the presence of serum, toxicity and/or immunogenicity problems (Cohen et al., Gene Ther. 7:1896-905, 2000). Hence, it is important to demonstrate anti-tumor efficacy of dual-agent nanoparticles in vivo. One objective of this Example is to establish the anti-tumor efficacy of dual-agent nanoparticles in a mouse xenograft model of drug resistant tumor. The Example is designed to test the hypothesis that dual-agent nanoparticles that demonstrate sustained cytotoxicity in vitro and enhanced tumor-targeting in vivo will result in regression of resistant tumor in vivo. The approach used is evaluation of dose dependency in tumor growth suppression following intravenous injection of dual-agent nanoparticles in mouse xenograft model of tumors overexpressing either P-gp or Hsp70. An optimized nanoparticle formulation based on the results in Examples 1 and 2 will be tested to determine the regression of drug-resistant tumor. A goal of this and the previous Examples is to establish a dose of dual-agent nanoparticles required for regression of drug resistant tumor.

Tumor model. MCF/Dox and MCF-7/HSP70 cells will be used to induce drug-resistant tumors in ovariectomized female NCRNU-M mice. Tumor induction will be as described before. One experiment will be performed for each cell type. When tumor sizes are between 100 mm³ and 400 mm³, animals will be injected with different treatments as described below.

Effect of dose. An objective of the Example is to determine the dose-dependency in tumor regression with dual-agent nanoparticles. A tumor will be considered as regressed if, at the end of the study, its volume is less than its pre-treatment levels. The optimal dose of siRNA and paclitaxel may be determined using a randomized complete factorial design. Each of the factors may be examined at three different dose levels, resulting in 9 treatment groups. Paclitaxel may be examined at 100, 200, and 400 μg, while siRNA may be examined at doses of 5, 10, and 20 μg. Paclitaxel dose was selected based on the fact that a dose of ˜7 ng/day/8 μg nanoparticles was effective in overcoming drug resistance in about 5×10³ MDR cells. This dose was escalated by a factor of 10³ to give the median in vivo dose for the 30-day study, because the number of tumor cells in the in vivo study is 10³ times higher than in the in vitro study. Thus, 7 ng×30×10^{3≈200} μg was chosen as the median dose.

Similarly, siRNA at a dose of 0.3 ng/day/8 μg was effective in overcoming drug resistance in about 5×10³ MDR cells. This dose was escalated by a factor of 10³ to give the median in vivo dose for the 30-day study. Different doses of siRNA and paclitaxel will be loaded in 8 mg of nanoparticles as described in Example 1. Tumor growth over a 30-day period will be used as the end point. Animals that develop tumors of 100-400 mm³ size will be randomized into nine different treatment groups (n=6 per group, 2 experiments, 108 animals), and treated with intravenous (tail vein) injection of different doses.

Differences in tumor volumes at the end of 30 days will be evaluated by ANOVA followed by Fisher's protected least significant difference test to evaluate pairwise comparisons among treatment groups. A probability level of p<0.05 will be considered significant. Surface response plots will be constructed as a function of various dose combinations to determine the optimal siRNA and paclitaxel dose for maximal tumor suppression using MINITAB™ software.

Effect of dual-agent nanoparticles on long-term animal survival. Another objective of the Example is to investigate the efficacy of dual-agent nanoparticles in effecting chronic tumor regression and enhancing animal survival. The siRNA and paclitaxel dose that demonstrated maximal tumor regression in the above dose study will be used in this part of the Example. The dosing frequency will be determined from Example 2. A second dose of the treatment will be given when the paclitaxel concentration in the tumor falls below 100 nM. Based on the calculations above, it is expected that the second dose will need to be administered about 30 days after the first dose. The efficacy of dual-agent nanoparticles in effecting tumor regression and prolonging animal survival will be compared with other controls (Table 3).

Tumors will be induced in as described above. Animals that develop at least 100 mm³ will be randomized into eight different treatment groups (n=6 per group, 2 experiments, total of 96 animals). Tumor bearing mice will be treated with single intravenous injection of different treatments in Hank's balanced salt solution as outlined in Table 3. The Kaplan-Meier method will be used to analyze the survival curves in tumor-bearing mice. The time-to-event data for animals that did not reach the target tumor volume, either because of long-term cure (defined as those animals that were still alive at the conclusion of the experiment whose tumors either completely regressed or did not reach the preset target volume) or early death/euthanasia because of treatment toxicity, tumor metastasis or tumor volumes larger than 2500 mm³ will be treated as censored data. Wilcoxon and log-rank tests will be used to compare different treatment groups.

TABLE 3
Effect of nanoparticles on tumor growth and animal survival

| Group | Animals | Protocols |
|---|---|---|
| 1 | 6 | Dual-agent nanoparticles |
| 2 | 6 | Nanoparticles containing only siRNA |
| 3 | 6 | Nanoparticles containing only paclitaxel |
| 4 | 6 | Nanoparticles containing only siRNA + paclitaxel in |
| | | Cremophor EL solution |
| 5 | 6 | Nanoparticles containing only paclitaxel + siRNA in |
| | | solution |
| 6 | 6 | Nanoparticles containing non-targeted siRNA + |
| | | paclitaxel |
| 7 | 6 | Paclitaxel in Cremophor EL solution |
| 8 | 6 | Vehicle control |

Expected Outcomes. Related in vitro studies show that dual-agent nanoparticles overcome drug resistance and sustain cytotoxicity in drug resistant tumor cells. It is, therefore, expected that animals treated with dual-agent nanoparticles will demonstrate sustained tumor regression and enhanced survival than animals in other groups. Animals in group 4 and 5 are expected to have lower tumor growth and survive better than animals in other control groups, because related preliminary studies show that drug/siRNA in nanoparticles results in reversal of drug resistance. Overall, it is expected that studies in Example 3 will provide preliminary data establishing the in vivo efficacy of dual-agent nanoparticles in drug-resistant tumors.

### Example 4

### Effect of Folic Acid or Biotin Conjugation on Nanoparticle Uptake in Cancer Cell Lines

Nanoparticles containing 6-coumarin as a fluorescent marker were formulated using a double emulsion-solvent evaporation technique. In brief, an aqueous solution of BSA (60 mg/mL) was emulsified in a polymer solution (180 mg in 6 mL of chloroform) containing 6-coumarin (100 μg) using a probe sonicator (55 Watts for 2 min; Sonicator® XL, Misonix, N.Y., USA). The water-in-oil emulsion thus formed was further emulsified into 50 mL of 2.5% w/v aqueous solution of PVA by sonication as above for 5 min to form a multiple water-in-oil-in-water emulsion. Following this, a diblock copolymer polylactide-polyethylene glycol conjugated to folic acid (PLA-PEG-folic acid) and/or PLA-PEG-biotin was introduced. The multiple emulsion was stirred for 18 h under ambient conditions followed by for 1 h in a desiccator under vacuum. Nanoparticles thus formed were recovered by ultracentrifugation (100,000 g for 20 min at 4° C.), washed two times to remove PVA, unentrapped BSA, and 6-coumarin, and then lyophilized for 48 h to obtain a dry powder.

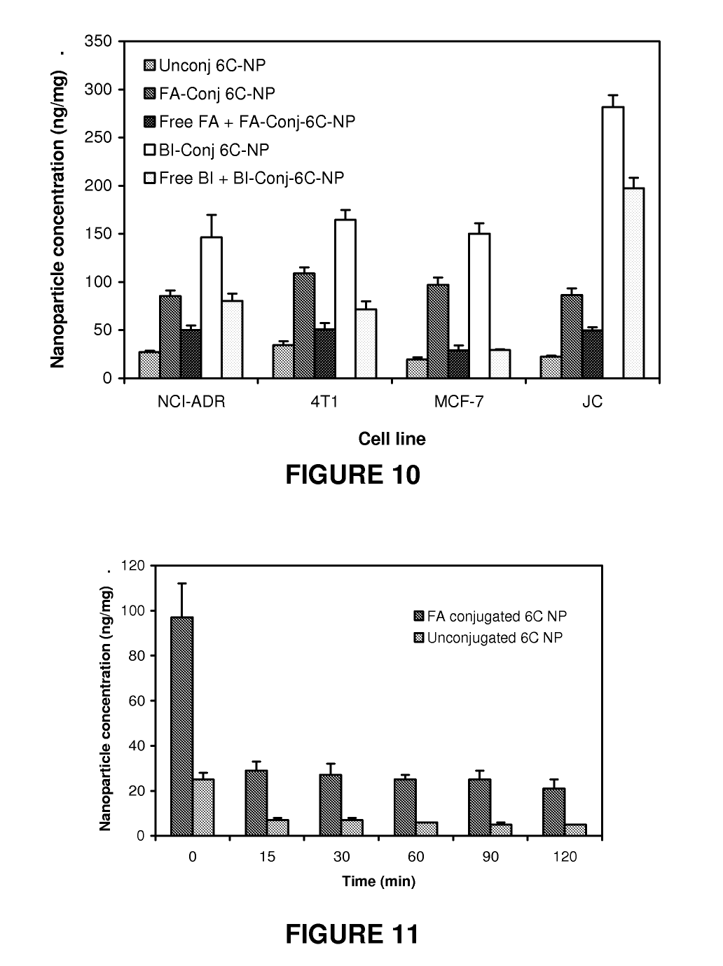

To study cell uptake, different cancer cells were seeded in 24-well plates at about 50,000 cells/well in 1 ml of growth medium. Cells were allowed to attach overnight and then treated with nanoparticles conjugated to folic acid (FA-Conj 6C-NP in the figure), nanoparticles conjugated to folic acid+excess free folic acid (Free FA+FA-Conj 6C-NP), nanoparticles conjugated to biotin (BI-Conj 6C-NP), nanoparticles conjugated to biotin+excess free biotin (Free BI+BI-Conj 6C-NP) or nanoparticles without folic acid or biotin on the surface (Unconj 6C-NP). Cells were then washed three times with phosphate-buffered saline (PBS, pH 7.4, 154 mM) and then lysed by incubating them with cell lysis buffer at 37° C. The cell lysates were processed to determine the nanoparticle levels by high-performance liquid chromatography (HPLC) as per our previously published method (Panyam et al, Int J. Pharm. 2003 Aug. 27; 262(1-2):1-11). Results are shown in FIG. 10, and were expressed as nanoparticle amount in μg per mg total cell protein.

### Example 5

### Effect of Folic Acid Conjugation on Nanoparticle Retention in NCl/ADR Cancer Cell Line

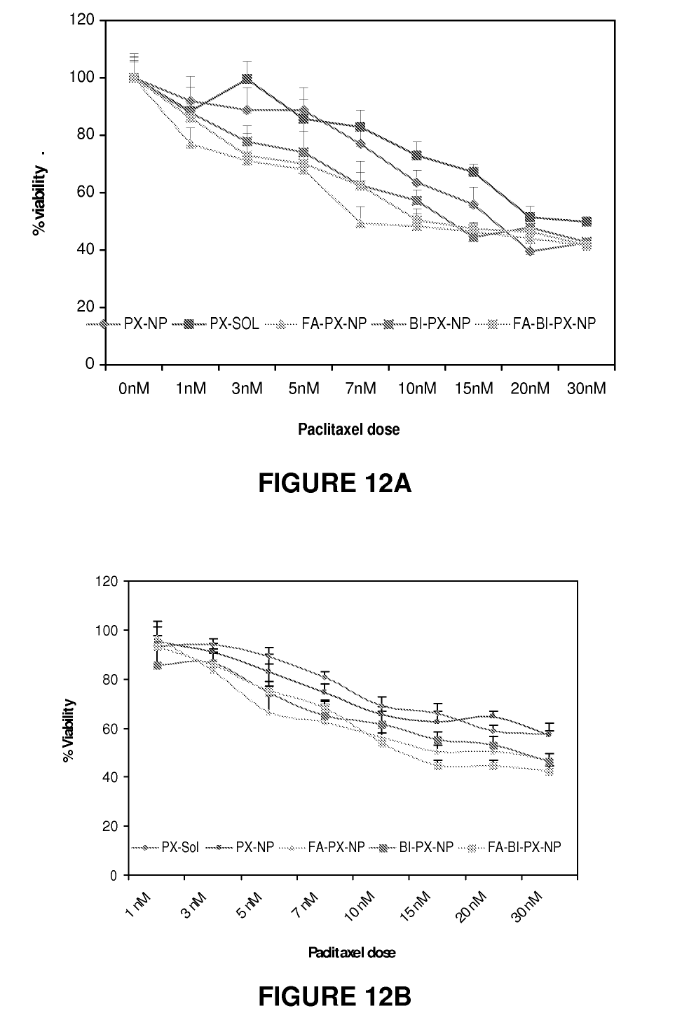

Nanoparticles containing 6-coumarin were prepared as described earlier. Nanoparticle retention in cells was followed by incubating the cells with nanoparticles for 1 h in regular growth medium followed by washing off of the uninternalized nanoparticles with PBS for two times. The intracellular nanoparticle level after the washing of the cells was taken as the zero time point value. The cells in other wells were then incubated with fresh growth medium. At different time intervals, the medium was removed, cells were washed twice with PBS and lysed, and the intracellular nanoparticle levels were analyzed to obtain the fraction of nanoparticles that were retained. The results are shown in FIG. 11.

### Example 6

### Effect of Folic Acid and Biotin Conjugation on In Vitro Cytotoxicity of Paclitaxel in Breast Cancer Cell Line MCF-7

Nanoparticles containing paclitaxel as a model anticancer drug were formulated using an emulsion-solvent evaporation technique. In brief, a polymer solution containing paclitaxel was emulsified into aqueous solution of PVA by sonication for 5 min to form a oil-in-water emulsion. Following this, we introduced a diblock copolymer polylactide-polyethylene glycol conjugated to folic acid (PLA-PEG-folic acid) and/or PLA-PEG-biotin. The emulsion was stirred for 18 h under ambient conditions followed by for 1 h in a desiccator under vacuum. Nanoparticles thus formed were recovered by ultracentrifugation (100,000 g for 20 min at 4° C.), washed two times to remove PVA, unentrapped paclitaxel, and then lyophilized for 48 h to obtain a dry powder.

For cytotoxicity studies, MCF-7 cells were seeded in 96-well plates at a seeding density of 5000 cells/well/0.1 ml medium, and allowed to attach overnight. Cells were then treated with medium containing paclitaxel in solution (PX-SOL), paclitaxel in nanoparticles without folic acid or biotin (PX-NP), paclitaxel in nanoparticles with folic acid (FA-PX-NP), paclitaxel in nanoparticles with biotin (BI-PX-NP), paclitaxel in nanoparticles with both folic acid and biotin (FA-BI-PX-NP). The medium was changed after 24 hrs, and no further dose of paclitaxel or verapamil was added.

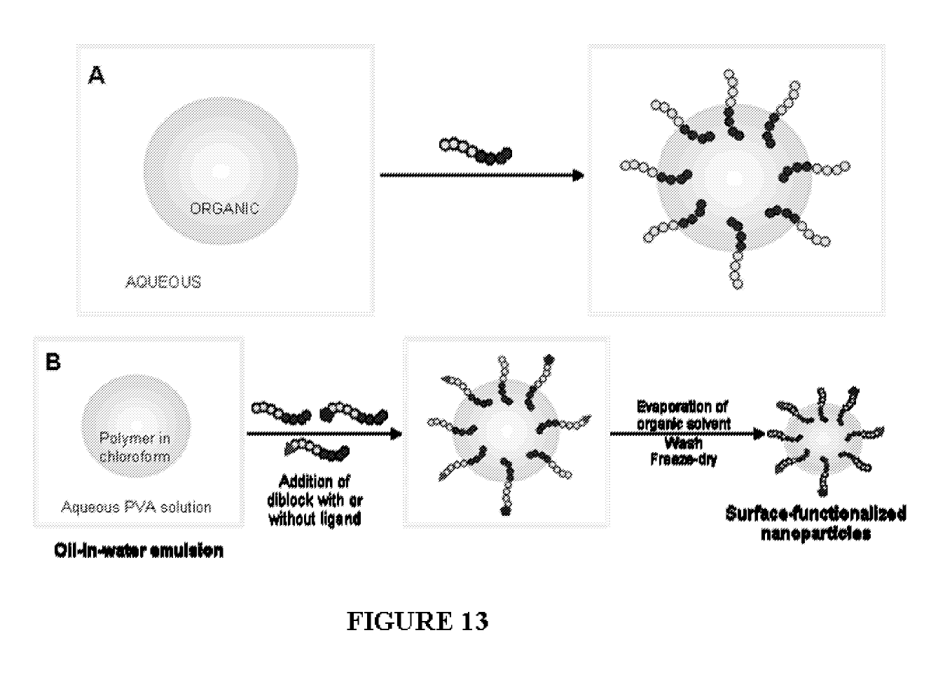

Cell viability was followed by MTS assay (CellTiter 96 Aqueous, Promega) over a period of 3 days. At different time intervals, the MTS assay reagent (20 μl) was added to each well, incubated for 120 min, and the absorbance was measured at 505 nm using a microplate reader (Molecular Devices, Kinetic microplate reader, Sunnyvale Calif.). In this assay, absorbance is proportional to number of viable cells. Untreated cells and empty nanoparticle-treated cells were used as controls. Results as shown in FIG. 12 A and 12B were presented as percentage viability compared to control.

### Example 7

### Interfacial Activity Assisted Surface Functionalization (IAASF)

This Example describes a novel interfacial activity assisted surface functionalization technique for polymeric nanoparticles. In summary, the technique utilizes the fact that the introduction of an amphiphilic diblock copolymer like polylactide-polyethylene glycol (PLA-PEG) in an oil/water system results in partitioning of PLA chain into the oil phase and PEG chain into the aqueous phase. This technique enabled the incorporation of multiple functional groups and tumor-targeting ligands on drug-loaded nanoparticles in a single step. Nanoparticles surface-functionalized with PEG, folic acid and biotin were able to improve paclitaxel delivery to tumor tissue, resulting in a significant inhibition of tumor growth in a mouse xenograft tumor model. Practical and industrial applicability of this technique are as follows.

An important goal in drug therapy is to enhance the availability of the drug at the site of action while minimizing drug exposure to non-target sites. Nanocarriers such as nanoparticles have emerged as versatile carrier systems for delivering small molecular weight drugs as well as macromolecular therapeutic agents to the tissue of interest. (Goldberg M, Langer R, Jia X, J Biomater Sci Polym Ed. 2007; 18(3):241-68). The use of biodegradable polymeric materials in nanoparticle fabrication allows for efficient encapsulation and controlled release of the therapeutic agent. Surface functionalization of nanocarriers with hydrophilic polymers such as polyethylene glycol and tissue-recognition ligands enables enhanced drug targeting. (van Vlerken L E, Vyas T K, Amiji M M, Pharm Res. 2007 August; 24(8):1405-14. Epub 2007 Mar. 29).

Prior art methods of incorporating targeting ligands on the surface of nanoparticles involve either physical adsorption (Cho et al., Macromol. Biosci. 5:512-519, 2005) or chemical conjugation of the ligand to pre-formed nanoparticles (Sahoo and Labhasetwar, Mol. Pharm. 2:373-83, 2005). Physical adsorption results in weak and temporary binding of the ligand on nanoparticle surface. The efficiency of ligand attachment is relatively low and frequently results in the aggregation of the carrier. Covalent chemical conjugation is not useful if the material used for nanoparticle fabrication lacks reactive functional groups or if the reaction conditions are detrimental to the payload in nanoparticles or to the targeting ligand. Further, chemical conjugation involves addition of pre-formed nanoparticles to a liquid reaction medium, which results in the leaching and loss of the payload from nanoparticles. Chemical coupling of the ligand to nanoparticles can be expensive and time consuming because the chemistry needs to be optimized for each nanoparticle-ligand combination. Current conjugation techniques are not suitable for incorporating multiple ligands on a single surface.

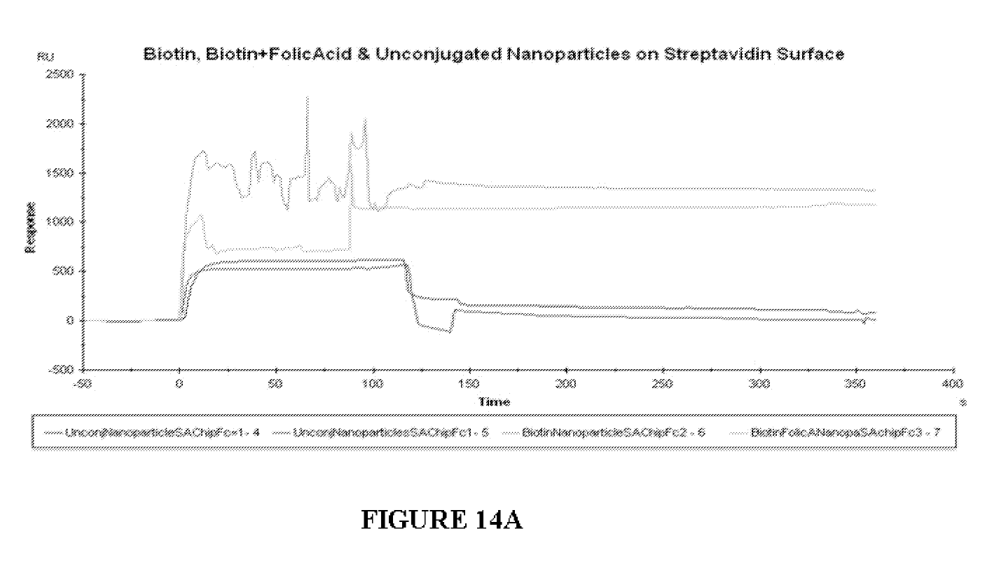

Described herein is a simple, interfacial activity-assisted method of nanoparticle surface functionalization. This method utilizes the fact that when an amphiphilic diblock copolymer is introduced into a biphasic (oil/water) system, the copolymer adsorbs at the interface. The hydrophobic block of the copolymer tends to partition into the oil phase while the hydrophilic block tends to remain in the aqueous phase (FIG. 13A). Most nanoparticles used in drug delivery are formulated using some modification of the emulsion solvent evaporation technique (Panyam J, Labhasetwar V, Adv Drug Deliv Rev. 2003 Feb. 24; 55(3):329-47). Polymer of interest is dissolved in an organic solvent like dichloromethane and this polymer solution is emulsified in an aqueous solution containing a surfactant such as polyvinyl alcohol. Removal of organic solvent from the system results in the formation of nanoparticles. A diblock copolymer like polylactide-polyethylene glycol (PLA-PEG) is introduced with or without a ligand conjugated to the PEG chain (PLA-PEG-ligand). This results in partitioning of polylactide block into the polymer containing oil phase and PEG-ligand block into the aqueous phase. Removal of the organic solvent results in the formation of nanoparticles with PEG or PEG-ligand on nanoparticle surface. Micelles formed due to the self-assembly of the PLA-PEG block copolymer are removed by extensive dilution and washing of the system. This method is referred to herein as Interfacial Activity Assisted Surface Functionalization (IAASF).

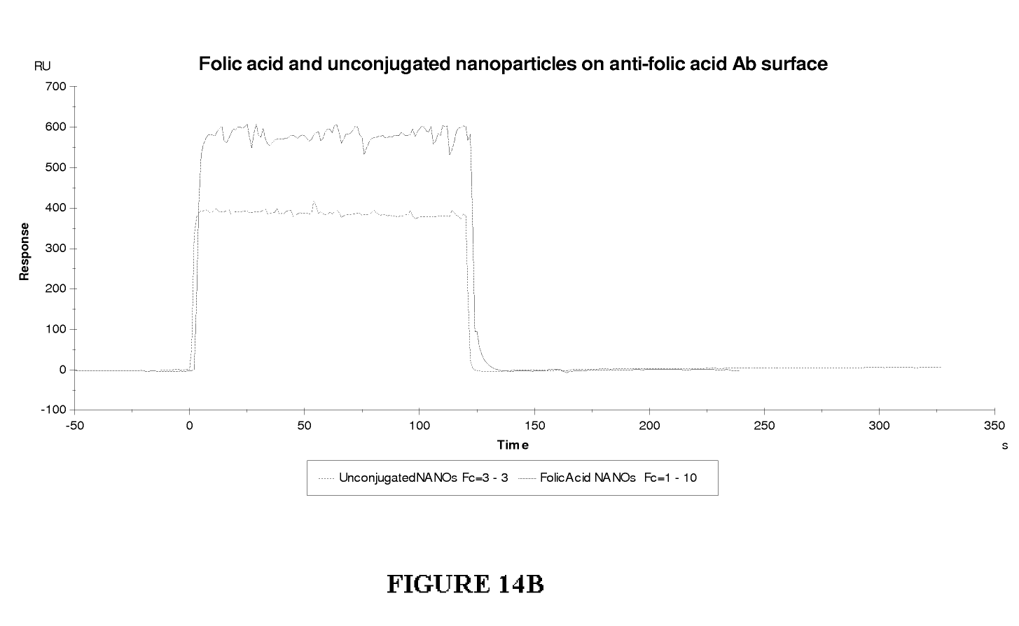

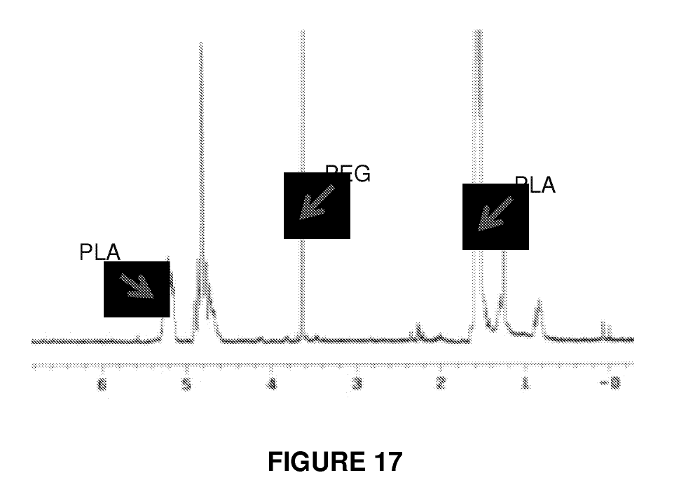

Nanoparticles were fabricated from a biodegradable polymer poly(D,L-lactide-co-glycolide) (PLGA) and surface functionalized with PEG, folic acid and/or biotin as targeting ligands (FIG. 13B). Incorporation of PLA-PEG segments along with the ligand(s) in nanoparticles was confirmed by proton NMR (FIG. 17). Presence of PEG and the ligands on the surface was confirmed by contact angle measurements (Table 2), and surface plasmon resonance (FIG. 14).

TABLE 4
Decrease in contact angle following incorporation
of PEG on nanoparticle surface

| Formulation | Contact angle (θ) |
|---|---|
| Unconjugated nanoparticles | 49 ± 5 |
| PEG conjugated nanoparticles | 33 ± 3 |

Decrease in the contact angle of water suggests that incorporation of PEG significantly increased the hydrophilicity of nanoparticle surface. This was expected, because PEG is more hydrophilic than PLGA. The decreased hydrophilicity of PEGylated nanoparticles is expected to contribute to the decreased biorecognition and increased circulation time of nanoparticles. Surface plasmon resonance studies indicated that not only were the ligands folic acid and biotin present on the surface of nanoparticles but were also available for binding. A significant difference in binding was observed for nanoparticles with and without ligands on the surface. For example, ˜20-fold increase in response units was observed for biotin-functionalized nanoparticles compared to non-functionalized nanoparticles (FIG. 14).

An important advantage of the IAASF technique is that it depends only on the interfacial activity of the block copolymer and the presence of a biphasic system. The method can thus be used potentially for a wide variety of polymers, therapeutic agents and targeting ligands. The composition of the diblock copolymer can altered to match the polymer used in nanoparticle fabrication. For example, PLGA can be replaced with other synthetic polymers such as polyanhydrides or polycaprolactone, while folic acid can be replaced with other ligands such as biotin (FIG. 2A). Further, this method can be used to incorporate reactive functional groups on nanoparticle surface for further chemical modifications. For example, nanoparticles can be surface functionalized with maleimide groups using PLA-PEG(maleimide) copolymer or with amino groups using PLA-PEG(NH₂) copolymer. These functionalities can then be used for incorporating peptide molecules or fluorophores on nanoparticle surface. For example, the maleimide functionality was used to incorporate cyclic RGD peptides on nanoparticle surface (not shown). Similarly, the amine functionality was used to conjugate fluorescein molecules on nanoparticle surface (FIG. 14B).

One advantage of IAASF method is that it enables the incorporation of multiple ligands and/or functional groups on nanoparticle surface in a single step. For example, addition of mixture of PLA-PEG-folic acid and PLA-PEG-biotin to the emulsion resulted in the incorporation of both folic acid and biotin on nanoparticle surface (FIG. 14). Surface plasmon resonance studies indicated that the presence of multiple ligands (for example, nanoparticles with both biotin and folic acid) on the surface resulted in slightly weaker binding for the individual ligands. For example, nanoparticles with biotin alone resulted in 1340 response units for binding with streptavidin while nanoparticles with both biotin and folic acid resulted in 1145 response units for binding with streptavidin.

Theoretically, the number of ligands that can be incorporated on nanoparticle surface is only limited by the total surface area available on each particle for ligand incorporation and by steric considerations. Quantitative assays of biotin and maleimide functional groups indicate that at least 4×10⁵ PEG molecules are introduced on each nanoparticle. Incorporation of multiple ligands on the surface would enable simultaneous targeting of multiple antigens and/or receptors in the target tissue. For example, simultaneous targeting of multiple components of the tumor tissue can be accomplished, such as the cancer cells, stroma and the vasculature, to improve targeting to tumor tissue.

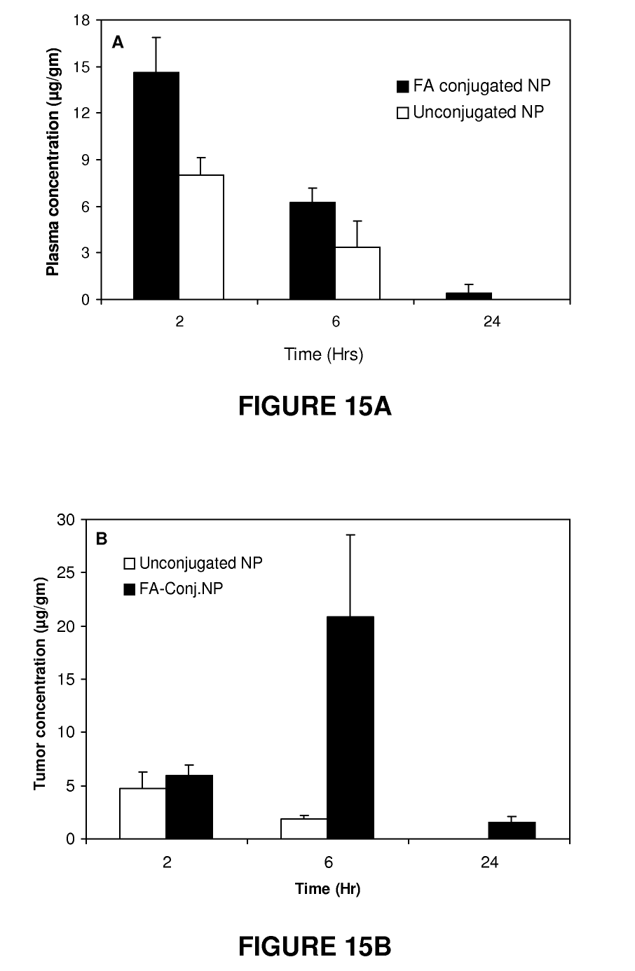

To determine whether the IAASF technique results in nanoparticles that function in vivo, nanoparticles were fabricated with different surface functionalizations and evaluated them for tumor-targeted drug delivery in mouse tumor models. Previous studies have shown that incorporation of PEG on nanoparticle surface prolongs the blood circulation time of nanoparticles and enables passive targeting of tumor tissue (Kommareddy S, Tiwari S B, Amiji M M, Technol Cancer Res Treat. 2005 December; 4(6):615-25). Previous studies have also shown that certain breast tumor cells overexpress folic acid and biotin receptors (Chavanpatil M D, Khdair A, Panyam J, J Nanosci Nanotechnol. 2006 September-October; 6(9-10):2651-63). Nanoparticles conjugated to folic acid (Hilgenbrink A R, Low P S, J Pharm Sci. 2005 October; 94(10):2135-46) or biotin (Lee E S, Na K, Bae Y H, Nano Lett. 2005 February; 5(2):325-9) target these tumor cells in vitro and in vivo. Fluorescently-labeled nanoparticles were fabricated with PEG and folic acid on the surface using the IAASF technique. Following intravenous administration of nanoparticles in Balb/C mice bearing JC tumors, the plasma and tumor concentrations of nanoparticle-associated fluorescent label were determined at different time intervals. PEG and folic acid-functionalized nanoparticles resulted in a significantly higher (P <0.05) plasma and tumor concentrations than non-functionalized nanoparticles (FIGS. 15A and 15B). Previous studies suggest that the surface functionalization of nanoparticles with PEG protects particles against opsonization and rapid systemic clearance.

The ability of surface functionalized nanoparticles to deliver a payload to the target tissue was evaluated. Paclitaxel, a microtubule stabilizing agent that is used extensively in the clinic against several types of cancer, was used as a model anticancer drug. Effect of a single-dose paclitaxel treatment on tumor growth was investigated in nude mice bearing MCF-7 xenografts. MCF-7 (breast carcinoma) cells are sensitive to paclitaxel and are known to overexpress both folate and biotin receptors. Paclitaxel-loaded nanoparticles were fabricated with PEG, folic acid and/or biotin on the surface using the IAASF technique. At the dose used (400 μg paclitaxel/animal), free paclitaxel and paclitaxel encapsulated in non-surface functionalized nanoparticles were only marginally effective. Incorporation of folic acid or biotin on the surface resulted in an improvement in therapeutic efficacy.

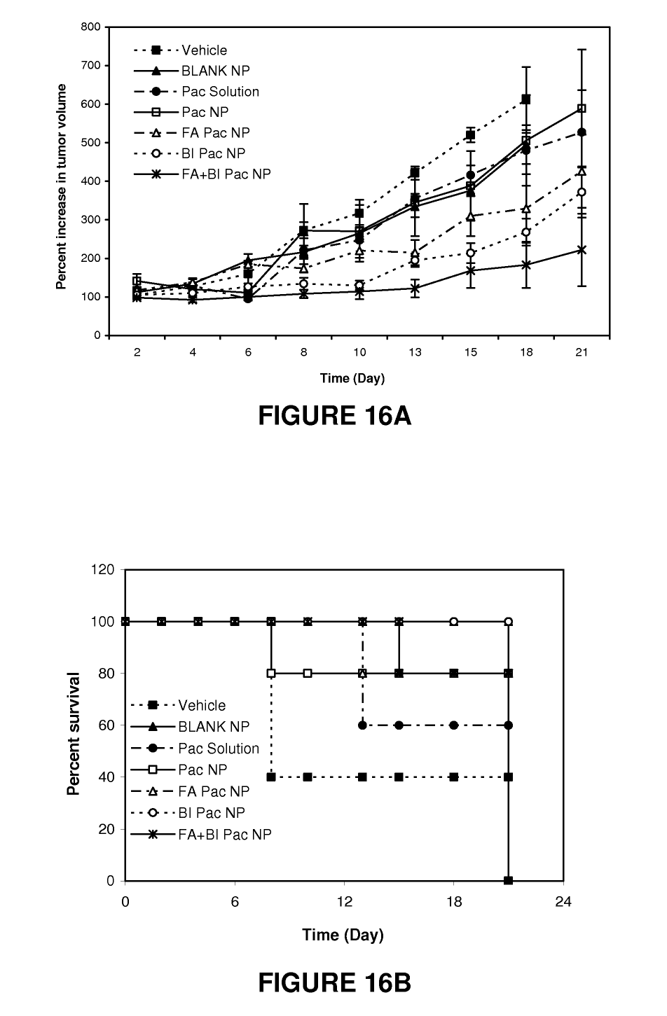

Treatment with nanoparticles that had both folic acid and biotin on the surface resulted in complete tumor regression in one animal and significant inhibition in tumor growth in other animals (FIG. 16A). Enhanced tumor inhibition was accompanied by increased survival in treated groups. At the end of 80 days post-treatment, 50% of animals that received folic acid and biotin functionalized nanoparticles survived while the survival rates were 40% and 20% in folic acid-functionalized nanoparticle group and biotin nanoparticle group, respectively. None of the animals survived in the other groups (FIG. 16B). Increased recognition and uptake by the tumor cells of drug-loaded nanoparticles that were functionalized with both biotin and folic acid could have contributed to the enhanced therapeutic efficacy of these nanoparticles.

In summary, this Example describes a novel surface-functionalization methodology that is adaptable to a wide variety of nanoparticle platforms, therapeutic agents and targeting ligands. The IAASF technique enables the incorporation of multiple surface functionalities in a single step. This new surface functionalization approach has industrial and clinical applicability for enabling the development of novel targeting strategies such as the use of multiple targeting ligands on a single surface for the delivery of drugs to the tissue of interest.

### Example 8

### c(RGD) Peptide Conjugation to Nanoparticles

Nanoparticles with maleimide groups on the surface were used for conjugating cRGD peptide on the surface. Nanoparticles with maleimide groups were prepared using the IAASF technique. Briefly, an aqueous solution of BSA was emulsified in PLGA polymer solution containing 6-coumarin using a probe sonicator. The water-in-oil emulsion thus formed was further emulsified into aqueous solution of polyvinyl alcohol by sonication as above to form a multiple water-in-oil-in-water emulsion. Following this, we introduced a diblock copolymer polylactide-polyethylene glycol with terminal maleimide functional group. The multiple emulsion was stirred for 18 h at room temperature followed by 1 h in a desiccator under vacuum. Nanoparticles thus formed were recovered by ultracentrifugation, washed two times, and then lyophilized for 48 h to obtain a dry powder. 40 mg of these nanoparticles were dispersed in 1 mL 0.05 M HEPES buffer containing 0.05M EDTA solution. 5 mg of c(RGD) peptide was dissolved in 200 μL 0.05 M HEPES+0.05M EDTA+0.005M Hydroxyl amine HCL solution. c(RGD) peptide solution was then added to the nanoparticle dispersion and incubated overnight at room temperature. This resulted in c(RGD) peptide conjugation to nanoparticles. Unconjugated peptide was removed by diluting nanoparticles in HEPES/EDTA buffer and repeated centrifugation. (Nano Letters 6:2427-2430 (2006))

### Example 9

### Fluorescein Isothiocyanate (FITC) Conjugation to Nanoparticles

Nanoparticles with amino groups on the surface were used for conjugating FITC on nanoparticle surface. Nanoparticles with amino groups were prepared using the IAASF technique. Briefly, an aqueous solution of BSA was emulsified in PLGA polymer solution containing 6-coumarin using a probe sonicator. The water-in-oil emulsion thus formed was further emulsified into aqueous solution of polyvinyl alcohol by sonication as above to form a multiple water-in-oil-in-water emulsion. Following this, we introduced a diblock copolymer polylactide-polyethylene glycol with terminal amine functional group. The multiple emulsion was stirred for 18 h at room temperature followed by 1 h in a desiccator under vacuum. Nanoparticles thus formed were recovered by ultracentrifugation, washed two times, and then lyophilized for 48 h to obtain a dry powder. 50 mg of these nanoparticles were dispersed in 500 mM carbonate buffer (pH 9.5). FITC (1:5 PEG-amine to FITC mole ratio) was dissolved in anhydrous DMSO. FITC solution was then added to the nanoparticle dispersion and stirred for 4 hrs at room temperature. This resulted in FITC conjugation to nanoparticles. Unconjugated FITC was removed by diluting nanoparticles in carbonate buffer and repeated centrifugation.

While the description above refers to particular embodiments of the present invention, it will be understood that many modifications may be made without departing from the spirit thereof. The presently disclosed embodiments are therefore to be considered in all respects as illustrative and not restrictive. All patents, patent applications, provisional applications, and publications referred to or cited herein are incorporated by reference in their entirety, including all figures and tables, to the extent they are not inconsistent with the explicit teachings of this specification.

We claim:

1. A method of treating a tumor in a subject, the method comprising contacting a subject in need thereof with a nanoparticle comprising at least one polymer and at least one therapeutic agent joined thereto, under suitable conditions such that at least one tumor-related effect occurs.

2. The method of claim 1 wherein the suitable conditions comprise a sustained time period of at least 1 day, at least 2 days, at least 5 days, at least 10 days, at least 20 days, at least 30 days, at least 45 days, and at least 60 days.

3. The method of claim 1 wherein the polymer is selected from the group consisting of: aliphatic polyesters; poly(glycolic acid); poly(lactic-co-glycolic acid); poly(caprolactone glycolide)); poly(lactic acid); polylactide (PLA); poly-L(lactic acid); poly-D Lactic acid; poly(caprolactone lactide); poly(lactide glycolide), poly(lactic acid ethylene glycol)); poly(ethylene glycol); poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides; poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; and biodegradable polyesters.

4. The method of claim 3 wherein the polymer comprises polylactide or poly(lactic-co-glycolic acid).

5. The method of claim 1 wherein the therapeutic agent is selected from the group consisting of: a polysaccharide, a peptide, a polypeptide, a nucleic acid, a vitamin, a mineral, a vaccine, a cytokine, an apoptotic agent, a cytotoxic agent, and a pharmaceutical drug.

6. The method of claim 5 wherein the therapeutic agent comprises paclitaxel, dexamethasone, a heat-shock protein, Bcl-2, Bcl-xl, or folic acid.

7. The method of claim 1 wherein the nanoparticle further comprises a detection agent joined thereto, wherein the detection agent is selected from the group consisting of: a magnetic compound, a paramagnetic compound, a fluorophore, a radioisotope, and an enzyme.

8. The method of claim 1 or claim 7 wherein the nanoparticle further comprises a functional group joined thereto, wherein the functional group is selected from the group consisting of: alkane, alkene, alkyne, amide, amine, imide, phosphine, phosphodiester, phosphonic acid, phosphate, sulfide, imidazole and oxazole.

9. The method of claim 1 wherein the tumor-related effect is selected from the group consisting of: decrease in tumor size, decrease in tumor cell proliferation, decrease in tumor cell metastasis, decrease in tumor vasculature, decrease in tumor angiogenesis, decrease in tumor blood flow, increase in cell differentiation, increase in tumor cell apoptosis, and increase in tumor cell necrosis.

10. A therapeutic composition comprising a nanoparticle, and at least one therapeutic agent joined thereto wherein the therapeutic agent confers a sustained biological or chemical effect over a time period.

11. The composition of claim 10, wherein the time period is selected from the group consisting of: at least 1 day, at least 2 days, at least 5 days, at least 10 days, at least 20 days, at least 30 days, at least 40 days, and at least 60 days.

12. The composition of claim 10 wherein the therapeutic agent is selected from the group consisting of: a polysaccharide, a peptide, a polypeptide, a nucleic acid, a vaccine, a cytokine, an apoptotic agent, a cytotoxic agent, a vitamin, a mineral, and a pharmaceutical drug.

13. The composition of claim 12 wherein the therapeutic agent comprises paclitaxel, dexamethasone, a heat-shock protein, Bcl-2, Bcl-xl, or folic acid.

14. The composition of claim 10 wherein the biological or chemical effect is selected from the group consisting of: decrease in tumor size, decrease in tumor cell proliferation, decrease in tumor cell metastasis, decrease in tumor vasculature, decrease in tumor angiogenesis, decrease in tumor blood flow, increase in cell differentiation, increase in tumor cell apoptosis, and increase in tumor cell necrosis.

15. The composition of claim 10 wherein the nanoparticle further comprises a detection agent joined thereto.

16. The composition of claim 15 wherein the detection agent is selected from the group consisting of: a magnetic compound, a paramagnetic compound, a fluorophore, a radio-isotope, and an enzyme.

17. The composition of claim 10 wherein the nanoparticle further comprises a functional group joined thereto.

18. The composition of claim 17 wherein the functional group is selected from the group consisting of: alkane, alkene, alkyne, amide, amine, imide, phosphine, phosphodiester, phosphonic acid, phosphate, sulfide, imidazole and oxazole.

19. A process of making a nanoparticle composition comprising a first step of emulsifying at least one first agent in the presence of at least one first polymer and at least one first solvent, thereby forming a water-in-oil emulsion; and a second step of emulsifying the water-in-oil emulsion with at least one second polymer, at least one second solvent, and at least one second agent wherein the first and second agents are the same or different and are selected from the group consisting of a therapeutic agent, a diagnostic agent, and a detection agent; thereby making a nanoparticle composition.

20. The process of claim 19, wherein the first polymer comprises poly(lactic co-glycolic acid) (PLGA), the first solvent comprises polyvinyl alcohol, the first agent comprises paclitaxel, dexamethasone, a heat-shock protein, Bcl-2, Bcl-xl, or folic acid, the second polymer comprises polylactide (PLA) or polyethylene glycol (PEG), the second solvent comprises methanol, and the second agent comprises folic acid.

21. A therapeutic composition comprising a nanoparticle, and at least one detection agent joined thereto wherein the detection agent confers a sustained biological or chemical effect over a time period.

22. The composition of claim 21 wherein the detection agent is selected from the group consisting of: a magnetic compound, a paramagnetic compound, a fluorophore, a radio-isotope, and an enzyme.
